(19) United States
(12) Patent Application Publication
(10) Pub. No.: US 2019/0119196 A1
(43) Pub. Date: Apr. 25, 2019

(54) COMPOUNDS FOR THE TREATMENT OF HYPERGLYCAEMIA

(71) Applicant: ATROGI AB, Stockholm (SE)

(72) Inventors: Benjamin Pelcman, Stockholm (SE); Tore Bengtsson, Vaxholm (SE)

(21) Appl. No.: 16/082,750

(22) PCT Filed: Mar. 7, 2017
(86) PCT No.: PCT/GB2017/050605

§ 371 Date: Sep. 6, 2018

(30) Foreign Application Priority Data
Mar. 7, 2016 (GB) 1603886.1
Feb. 6, 2017 (GB) 1701928.2

(51) Int. Cl. C07C 215/60; A61K 45/06; A61K 31/138; C07C 215/68

(52) U.S. Cl. CPC C07C 215/60; A61K 45/06; A61K 31/138; C07C 2603/74; C07C 2601/02; C07C 2601/14; C07C 2601/08; C07C 215/68

(57) ABSTRACT

There is herein provided a compound of formula (I) or a pharmaceutically acceptable salt thereof, for use in the treatment of hyperglycaemia or a disorder characterized by hyperglycaemia, such as type 2 diabetes, wherein X, R¹, R², R³ and n have meanings as provided in the description.

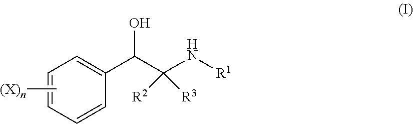

### FIELD OF THE INVENTION

The present invention relates to novel compounds and compositions, and their use in the treatment of hyperglycaemia and disorders characterised by hyperglycaemia, such as type 2 diabetes. In particular, the invention relates to novel compounds, compositions and methods for the treatment of conditions such as type 2 diabetes through activation of the β₂-adrenergic receptor. Importantly, such compounds are thought to have a beneficial side-effect profile as they do not exert their effect through significant cAMP release.

### BACKGROUND OF THE INVENTION

The listing or discussion of an apparently prior-published document in this specification should not necessarily be taken as an acknowledgement that the document is part of the state of the art or is common general knowledge.

Hyperglycaemia, or high blood sugar is a condition in which an excessive amount of glucose circulates in the blood plasma. If not treated, hyperglycaemia can be a serious problem, potentially developing into life-threatening conditions such as ketoacidosis. For example, chronic hyperglycemia may cause injury to the heart, and is strongly associated with heart attacks and death in subjects with no coronary heart disease or history of heart failure. There are various causes of hyperglycaemia, including diabetes and severe insulin resistance.

Severe insulin resistance (SIR) is a condition wherein the patent experiences very low levels of (or, in extreme cases, no significant) response to insulin. There are several syndromes characterized by SIR, including Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndrome, pseudoacromegaly, and lipodystrophy. The majority of these conditions have genetic causes, such as mutations in the insulin receptor gene. The prevalence for Donohue's syndrome, Rabson-Mendenhall syndrome and Type A syndrome of insulin resistance, has been reported to vary from about 50 reported cases to 1 in 100,000. However, since some diseases are severe and extremely rare, it is likely that many patients do not get diagnosed before they die, particularly in less developed areas of the world. Thus, the exact number of patients with these syndromes is difficult to assess.

The current standard for hyperglycaemia treatment in patients having SIR is a controlled diet, supplemented with drugs affecting insulin receptor sensitivity, such as metformin, or insulin supplement. However, particularly for disorders caused by mutations in the insulin receptor gene, this treatment is not sufficiently effective and ultimately proves unsuccessful.

Diabetes comprises two distinct diseases, type 1 (or insulin-dependent diabetes) and type 2 (insulin-independent diabetes), both of which involve the malfunction of glucose homeostasis. Type 2 diabetes affects more than 400 million people in the world and the number is rising rapidly. Complications of type 2 diabetes include severe cardiovascular problems, kidney failure, peripheral neuropathy, blindness and, in the later stages of the disease, even loss of limbs and, ultimately. Type 2 diabetes is characterized by insulin resistance in skeletal muscle and adipose tissue, and there is presently no definitive cure. Most treatments used today are focused on remedying dysfunctional insulin signalling or inhibiting glucose output from the liver but many of those treatments have several drawbacks and side effects. There is thus a great interest in identifying novel insulin-independent ways to treat type 2 diabetes.

In type 2 diabetes, the insulin-signalling pathway is blunted in peripheral tissues such as adipose tissue and skeletal muscle. Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (P13K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

It should be noted that skeletal muscles constitute a major part of the body weight of mammals and have a vital role in the regulation of systemic glucose metabolism, being responsible for up to 85% of whole-body glucose disposal. Glucose uptake in skeletal muscles is regulated by several intra- and extracellular signals. Insulin is the most well studied mediator but others also exist. For example, AMP activated kinase (AMPK) functions as an energy sensor in the cell, which can increase glucose uptake and fatty acid oxidation. Due to the great influence skeletal muscles have on glucose homeostasis it is plausible that additional mechanisms exists. In the light of the increased prevalence of type 2 diabetes, it is of great interest to find and characterize novel insulin-independent mechanisms to increase glucose uptake in muscle cells.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake, glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane α-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and α_{1D} subtypes while α₂-ARs are divided into α_{2A}, α_{2B} and α_{2C}. The β-ARs are also divided into the subtypes β₁, β₂, and β₃, of which β₂-AR is the major isoform in skeletal muscle cells. ARs are G protein coupled receptors (GPCRs) that signal through classical secondary messengers such as cyclic adenosine monophosphate (cAMP) and phospholipase C (PLC).

Many effects occurring downstream of ARs in skeletal muscles has been attributed to classical secondary messenger signaling, such as increase in cAMP levels, PLC activity and calcium levels. Stimulation involving the classical secondary messengers has many effects in different tissues. For example, it increases heart rate, blood flow, airflow in lungs and release of glucose from the liver, which all can be detrimental or be considered unwanted side effects if stimulation of ARs should be considered as a type 2 diabetes treatment. Adverse effects of classical AR agonists are, for example, tachycardia, palpitation, tremor, sweats, agitation and increased glucose levels in the blood (glucose output from the liver). It would thus be beneficial to be able to activate ARs without activating these classical secondary messengers, such as cAMP, to increase glucose uptake in peripheral tissues without stimulating the unwanted side effects.

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signaling increases glucose uptake is mainly via GLUT4 translocation from intracellular storage to the plasma membrane. It is known that GLUT4 translocation is induced by stimulation of the β₂-adrenergic receptor.

Thus, a possible treatment of a condition involving dysregulation of glucose homeostasis or glucose uptake in a mammal, such as type 2 diabetes, would involve the activation of the β₂-adrenergic receptor leading to GLUT4 translocation to the plasma membrane and promotion of glucose uptake into skeletal muscle leading to normalization of whole body glucose homeostasis. In addition, it would be advantageous if the treatment does not involve signalling through cAMP as this would lead to a favourable side-effect profile.

The vasodilator 4-(2-(butylamino)-1-hydroxyethyl)phenol, which has been used in the treatment of peripheral vascular disorders, has been found to initially increase blood sugar and has been contraindicated in diabetes and pre-diabetes (see Unger, H., Zeitschrift für die Gesamte Innere Medizin and Ihre Grenzgebiete, 16, 742 (1961)).

### Description of the Invention

We have now surprisingly found that certain β-hydroxyethylamines acting as agonists at the β₂-adrenergic receptor increase glucose uptake in skeletal muscle.

In addition, we have found that this effect is not mediated through significant cAMP release, such that many of the commonly described side effects seen with traditional β₂-adrenergic agonists (e.g. tachycardia, palpitation, tremor, sweats, agitation, and the like) can be reduced.

The use of such compounds in medicine represents a promising strategy for the treatment of conditions characterized by high blood sugar levels (i.e. hyperglycaemia), such as type 2 diabetes.

Compounds for Medical Use

In a first aspect of the invention, there is provided a compound of formula I

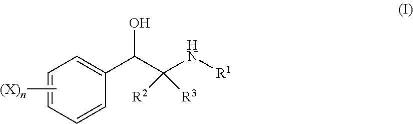

or a pharmaceutically acceptable salt thereof, for use in the treatment of hyperglycaemia or a disorder characterized by hyperglycaemia, wherein:

R¹ represents C₄₋₁₂ alkyl optionally substituted by one or more halo;

R² and R³ each independently represent H or C₁₋₃ alkyl optionally substituted by one or more halo;

or R² and R³ may be linked together to form, together with the carbon atom to which they are attached, a 3- to 6-membered ring, which ring optionally is substituted by one or more groups independently selected from halo and C₁ alkyl optionally substituted by one or more halo;

each X independently represents halo, R^{a}, —CN, —N₃, —N(R^{b})R^{c}, —NO₂, —ONO₂, —OR^{d}, —S(O)ₚR^{e} or —S(O)_{q}N(R^{f})R^{g};

R^{a} represents C₁₋₆ alkyl optionally substituted by one or more groups independently selected from G;

each R^{b}, R^{c}, R^{d}, R^{e}, R^{f} and R^{g} independently represents H or C₁₋₆ alkyl optionally substituted by one or more groups independently selected from G;

or alternatively any of R^{b} and R^{c} and/or R^{f} and R^{g} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;

G represents halo, —CN, —N(R^{a1})R^{b1}, —OR^{c1}, —S(O)ₚR^{d1}, —S(O)_{q}N(R^{e1})R^{f1} or ═O;

each R^{a1}, R^{b1}, R^{c1}, R^{d1}, R^{e1} and R^{f1} independently represents H or C₁₋₆ alkyl optionally substituted by one or more halo;

or alternatively any of R^{a1} and R^{b1} and/or R^{e1} and R^{f1} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;

n represents 0 to 5;

each p independently represents 0, 1 or 2; and

each q independently represents 1 or 2;

which compounds (including pharmaceutically acceptable salts) may be referred to herein as the “compounds of the invention”.

In an alternative first aspect of the invention, there is provided the use of a compound of formula I, or a pharmaceutically acceptable salt thereof, in the manufacture of a medicament for use in the treatment of hyperglycaemia or a disorder characterized by hyperglycaemia.

In a further alternative first aspect of the invention, there is provided a method of treating hyperglycaemia or a disorder characterized by hyperglycaemia comprising administering to a patient in need thereof a therapeutically effective amount of a compound of formula I, or a pharmaceutically acceptable salt thereof.

For the avoidance of doubt, the skilled person will understand that references herein to compounds of particular aspects of the invention (such as the first aspect of the invention, e.g. compounds of formula I) will include references to all embodiments and particular features thereof, which embodiments and particular features may be taken in combination to form further embodiments.

Unless indicated otherwise, all technical and scientific terms used herein have the same meaning as commonly understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds of the first aspect of the invention may exist as solids, and thus the scope of the invention includes all amorphous, crystalline and part crystalline forms thereof, and may also exist as oils. Where compounds of the first aspect of the invention exist in crystalline and part crystalline forms, such forms may include solvates, which are included in the scope of the invention. Compounds of the first aspect of the invention may also exist in solution.

Compounds of the first aspect of the invention may contain double bonds and may thus exist as E (entgegen) and Z (zusammen) geometric isomers about each individual double bond. All such isomers and mixtures thereof are included within the scope of the invention.

Compounds of the first aspect of the invention may also exhibit tautomerism. All tautomeric forms and mixtures thereof are included within the scope of the invention.

Compounds of the first aspect of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired optical isomers may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As used herein, references to halo and/or halogen groups will each independently refer to fluoro, chloro, bromo and iodo (for example, fluoro (F) and chloro (Cl)).

Unless otherwise specified, C_{1-z} alkyl groups (where z is the upper limit of the range) defined herein may be straight-chain or, when there is a sufficient number (i.e. a minimum of three) of carbon atoms, be branched-chain, and/or cyclic (so forming a C_{3-z}-cycloalkyl group). When there is a sufficient number (i.e. a minimum of four) of carbon atoms, such groups may also be part cyclic. Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic. Such alkyl groups may also be saturated or, when there is a sufficient number (i.e. a minimum of two) of carbon atoms, be unsaturated (forming, for example, a C_{2-z} alkenyl or a C_{2-z} alkynyl group). Particular alkyl groups that may be mentioned include saturated alkyl groups.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur).

For the avoidance of doubt, references to polycyclic (e.g. bicyclic or tricyclic) groups (e.g. when employed in the context of cycloalkyl groups) will refer to ring systems wherein at least two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, and may also refer to groups in which two non-adjacent atoms are linked by an alkylene group, which later groups may be referred to as bridged.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more X groups are present, those X groups may be the same or different. Similarly, where two or more X groups are present and each represent halo, the halo groups in question may be the same or different. Likewise, when more than one R^{a} is present and each independently represents C₁₋₆ alkyl substituted by one or more G group, the identities of each G are in no way interdependent.

The skilled person will appreciate that compounds of the invention that are the subject of this invention include those that are stable. That is, compounds of the invention include those that are sufficiently robust to survive isolation, e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

In certain embodiments of the first aspect of the invention, there is provided the compounds of the invention (i.e. the compound of formula I) wherein when n represents 1 and the X substituent is located in the 4-position of the benzene ring (i.e. the 4-position of the essential benzene ring relative to the point of substitution by the essential substituent) and represents —OR^{d}, then R^{d} represents H.

In alternative embodiments of the first aspect of the invention, there is provided the compounds of the invention wherein, when n represents at least 1 and an X substituent is present in the 4-position of the benzene ring (i.e. the 4-position of the essential benzene ring relative to the point of substitution by the essential substituent), the X substituent in the 4-position represents —N(R^{b})R^{c} or —OR^{d}, wherein R^{b}, R^{c} and R^{d} represents H.

In further alternative embodiments of the first aspect of the invention, there is provided the compounds of the invention wherein, when n represents at least 1 and an X substituent is present in the 4-position of the benzene ring (i.e. the 4-position of the essential benzene ring relative to the point of substitution by the essential substituent), the X substituent in the 4-position represents —OR^{d}, particularly wherein R^{d} represents H.

In particular embodiments of the first aspect of the invention, when n represents at least 1 and an X substituent is present in the 4-position of the benzene ring (i.e. the 4-position of the essential benzene ring relative to the point of substitution by the essential substituent), the X substituent in the 4-position does not represent halo.

In more particular embodiments of the first aspect of the invention, when n represents at least 1 and an X substituent is present in the 4-position of the benzene ring (i.e. the 4-position of the essential benzene ring relative to the point of substitution by the essential substituent), the X substituent in the 4-position does not represent chloro.

In more particular embodiments of the first aspect of the invention, n represents at least 1 and an X substituent is present in the 4-position of the benzene ring (i.e. the 4-position of the essential benzene ring relative to the point of substitution by the essential substituent), wherein the X substituent in the 4-position represents —OH.

In certain embodiments of the first aspect of the invention, there is provided the compounds of the invention (i.e. the compound of formula I) wherein when n represents 1 and the X substituent is located in the 3-position of the benzene ring (i.e. the 3-position of the essential benzene ring relative to the point of substitution by the essential substituent) and represents halo (e.g. Cl) or —OR^{d}, wherein R^{d} represents H.

In alternative embodiments of the first aspect of the invention, there is provided the compounds of the invention wherein, when n represents at least 1 and an X substituent is present in the 3-position of the benzene ring (i.e. the 3-position of the essential benzene ring relative to the point of substitution by the essential substituent), the X substituent in the 3-position represents halo (e.g. CI) or —OR^{d}, wherein R^{d} represents H.

Accordingly, in preferred embodiments, wherein n represents 1, X represents —OH and may be located in the 3-position or the 4-position of the benzene ring.

In particular embodiments of the first aspect of the invention, R¹ represents C4-10 alkyl optionally substituted by one or more F, such as C₄₋₈ alkyl optionally substituted by one or more F.

In more particular embodiments, R¹ represents C₄₋₈ alkyl optionally substituted by two or three F, such as C₄₋₆ alkyl optionally substituted by three F (e.g. where the three F are attached to the terminal carbon of the C₄₋₆ alkyl, e.g. 4,4,4-trifluorobutyl).

In more particular embodiments, R¹ represents C₄₋₁₀ alkyl, such as C₄₋₈ alkyl.

In alternative embodiments, R¹ represents C₄₋₁₂ alkyl.

In further embodiments, R¹ represents C₄₋₇ alkyl (e.g. C₄₋₅ alkyl).

In particular embodiments, the R¹ represents C₄₋₁₂ alkyl (e.g. C₄₋₇ alkyl, such as C₄₋₅ alkyl) wherein the carbon bound to the essential —NH— group is unbranched, e.g. represented by a —CH₂— moiety.

Particular R¹ groups that may be mentioned include those in which the alkyl group (for example, the C₄₋₁₂ alkyl group e.g. the C₄₋₁₀ alkyl, such as C₄₋₈ alkyl) is linear or part-cyclic (particularly part-cyclic such that the carbon bound to the essential —NH— group is unbranched, e.g. —CH₂—).

Other particular R¹ groups that may be mentioned include those in which the alkyl group (e.g. the C₄₋₁₀ alkyl, such as C₄₋₈ alkyl) is linear (e.g. n-butyl, n-hexyl or n-octyl).

Yet more particular R¹ groups that may be mentioned include those in which the alkyl group represents C₄₋₁₂ alkyl (e.g. C₄₋₇ alkyl, such as C₄₋₅ alkyl) wherein the carbon bound to the essential —NH— group is substituted by one substituent, e.g. represented by a —CH(R²)— moiety, wherein R² represents C₁₋₂ alkyl (e.g. methyl), e.g. wherein R² represents 2-pentyl.

In certain embodiments, R¹ represents n-butyl, sec-butyl, tent-butyl, 2-pentyl, cyclopentyl, —CH₂-cyclopropyl, —(CH₂)₂-cyclopropyl, n-hexyl, —(CH₂)₃-cyclopropyl, —CH₂-cyclohexyl, n-octyl, —(CH₂)₂-cyclohexyl, —(CH₂)₃-cyclohexyl, 4,4,4-trifluorobutyl or 1-adamantyl (e.g. n-butyl, tert-butyl or cyclopropylmethyl).

In further certain embodiments, R¹ represents n-butyl, sec-butyl, tent-butyl, 2-pentyl, cyclopentyl, —CH₂-cyclopropyl, —CH₂-cyclohexyl or 1-adamantyl (e.g. n-butyl, tent-butyl or cyclopropylmethyl).

In yet further embodiments, R¹ represents n-butyl, sec-butyl, 2-pentyl, cyclopentyl, —CH₂-cyclopropyl, —(CH₂)₂-cyclopropyl, n-hexyl, —(CH₂)₃-cyclopropyl, —CH₂-cyclohexyl or n-octyl, —(CH₂)₂-cyclohexyl, —(CH₂)₃-cyclohexyl or 1-adamantyl (e.g. n-butyl or cyclopropylmethyl).

In yet further embodiments, R¹ represents n-butyl, 2-pentyl, —CH₂-cyclopropyl, —(CH₂)₂-cyclopropyl, n-hexyl, —(CH₂)₃-cyclopropyl, —CH₂-cyclohexyl, n-octyl, —(CH₂)₂-cyclohexyl or —(CH₂)₃-cyclohexyl. In a particular embodiment, R¹ may represent —CH₂-cyclopropyl, —(CH₂)₂-cyclopropyl, —(CH₂)₃-cyclopropyl, —CH₂-cyclohexyl, —(CH₂)₂-cyclohexyl or —(CH₂)₃-cyclohexyl. In a further embodiment, R¹ may represent n-butyl or 2-pentyl.

In yet more particular embodiments, R¹ represents n-butyl, —CH₂-cyclopropyl, or —CH₂-cyclohexyl. In a particular embodiment, R¹ may represent —CH₂-cyclopropyl. In a further embodiment, R¹ may represent n-butyl.

In particular embodiments, R¹ does not represent tent-butyl.

In particular embodiments of the first aspect of the invention, each R² and R³ independently represents H or C₁₋₂ alkyl (e.g. methyl).

The skilled person will understand that the prefixes “n-”, “sec-”and “tert-”when applied to alkyl groups indicate the terms “normal”, “secondary” and “tertiary”. The term “normal” indicates a linear alkyl group where the point of attachment of the group to the rest of a molecule is through a carbon atom at the end of the carbon chain and thus that that carbon atom is bound to one other carbon atom. The term “secondary” indicates that the point of attachment of the rest of the molecule to the alkyl group is through a carbon atom adjacent to the end of the carbon chain and thus that that carbon is itself bound to two other carbon atoms. The term “tertiary” indicates that the point of attachment of the alkyl group to the rest of a molecule is through a carbon atom that is bound to three other carbon atoms.

In further embodiments of the first aspect of the invention, each R² and R³ independently represents H, methyl or ethyl (e.g. methyl).

In more particular embodiments, R² represents H and R³ represents H or methyl.

In yet more particular embodiments, R² and R³ each represent H.

In certain embodiments of the compounds of the invention, each X independently represents halo, R^{a}, —CN, —N₃, —N(R^{b})R^{c}, —NO₂ or —OR^{d}, wherein R^{a} represents C₁₋₄ alkyl optionally substituted by one or more F, and wherein R^{b}, R^{c} and R^{d} each independently represent H or C₁₋₄ alkyl optionally substituted by one or more F.

For example, each X may independently represent halo, R^{a}, —CN, —N₃, —N(R^{b})R^{c}, —NO₂ or —OR^{d}, wherein R^{a} represents C₁₋₄ alkyl optionally substituted by one or more F, and R^{b}, R^{c} and R^{d} each independently represent H or C₁₋₄ alkyl optionally substituted by one or more F.

In further embodiments, each X may independently represent halo, R^{a}, —CN, —N₃, —NO₂ or —OR^{d},wherein R^{a} represents C₁₋₄ alkyl optionally substituted by one or more F, and R^{d} each independently represent H or C₁₋₄ alkyl optionally substituted by one or more F.

In more particular embodiments, each X independently represents F, Cl, R^{a}, —NH₂, —CN or —OH, wherein R^{a} represents C₁₋₄ alkyl (e.g. C₁₋₂ alkyl) optionally substituted by one or more F (for example R^{a} represents —CHF₂ or —CF₃ (e.g. —CF₃)).

In yet more particular embodiments, each X independently represents F, Cl, R^{a}, —NH₂ or —OH, wherein R^{a} represents C₁₋₂ alkyl optionally substituted by one or more F (for example R^{a} represents —CHF₂ or —CF₃ (e.g. —CF₃)).

In further particular embodiments, each X independently represents F, Cl, R^{a}, or —OH, wherein R^{a} represents C₁₋₂ alkyl optionally substituted by one or more F.

In yet further particular embodiments, each X independently represents CI, —NH₂, —CF₃ or —OH. In a further embodiment, each X independently represents —OH.

In some embodiments of the compounds of the invention, n represents 0, 1, 2 or 3 (for example 1 or 2, e.g. 1).

In certain embodiments of the compounds of the invention, n represents 0, 1 or 2 (e.g. 0 or 1).

In other embodiments of the compounds of the invention, n represents 1, 2 or 3 (e.g. 1 or 2).

In certain embodiments, wherein n represents 3, each X independently represents halo (e.g. F or Cl, such as F), —NH₂, —CF₃ or —OH. In such embodiments, X groups may be located in the 3-, 4- and 5-positions of the essential benzene ring.

In certain embodiments, wherein n represents 2, each X independently represents F, Cl, —NH₂, or —OH. In such embodiments, the X groups may be located in the 3- and 4-positions, or the 3- and 5-positions of the essential benzene ring.

In certain embodiments, wherein n represents 2, each X independently represents Cl or —OH. In such embodiments, the X groups may be located in the 3- and 4-positions, or the 3- and 5-positions of the essential benzene ring.

In certain embodiments, wherein n represents 2, each X independently represents F, Cl, —CF₃ or —NH₂. In such embodiments, the X groups may be located in the 3- and 4-positions, or the 3- and 5-positions of the essential benzene ring.

In further embodiments:

n represents 2 or 3 and/or (e.g. and) each X independently represents halo (e.g. F or Cl, such as F), —NH₂, —CF₃ or —OH, particularly where such X groups are located in the 3-, 4- and 5-positions of the essential benzene ring.

In further embodiments, there is provided a compound of formula I, or a pharmaceutically acceptable salt thereof, wherein the essential benzene ring is unsubstituted in the 2- and 6-positions.

In particular embodiments of the first aspect of the invention:

each X independently represents halo, R^{a} or —OR^{d};

R^{a} represents C₁₋₄alkyl optionally substituted by one or more F;

R^{d} represents H or C₁₋₄alkyl optionally substituted by one or more F; and/or (e.g. and) n represents 0, 1, 2 or 3.

In more particular embodiments:

each X independently represents F, Cl, R^{a} or −OH;

R^{a} represents C₁₋₂alkyl optionally substituted by one or more F; and/or (e.g. and) n represents 0, 1 or 2 (e.g. 1 or 2).

In yet more particular embodiments:

each X independently represents F, Cl, methyl or —OH;

n represents 1 or 2 (e.g. 1); and/or (e.g. and) at least one X is in the 3- or in the 4-position on the phenyl group to which it is attached.

Examples of more particular embodiments include those wherein:

X independently represents Cl or —OH which substituents are in the 3- and 4-position on the phenyl group to which they are attached; and

n represents 2.

Other examples of more particular embodiments include those wherein:

X represents F, Cl, R^{a} or —OH in the 3-position on the phenyl group to which it is attached, wherein R^{a} represents C₁₋₂ alkyl optionally substituted by one or more F (for example, R^{a} may represent —CF₃ or —CH F₂ (e.g. —CHF₂)) ; and

n represents 1.

Yet more examples of more particular embodiments include those wherein:

X represents Cl or —OH (e.g., —OH) in the 3-position on the phenyl group to which it is attached; and

n represents 1.

Other examples of more particular embodiments include those wherein:

X represents F, Cl, R^{a} or —OH in the 4-position on the phenyl group to which it is attached, wherein R^{a} represents C₁₋₂ alkyl optionally substituted by one or more F (for example R^{a} may represent —CF₃ or —CHF₂ (e.g. —CHF₂)); and

n represents 1.

Yet more examples of more particular embodiments include those wherein:

X represents Cl or —OH (e.g. —OH) in the 4-position on the phenyl group to which it is attached; and n represents 1.

For example, in a particular embodiment of the first aspect of the invention:

R¹ represents n-butyl or cyclopropylmethyl;

R² and R³ represent H;

n represents 1; and

X represents —OH and is in the 4-position on the phenyl group to which it is attached.

In particular embodiments, where compounds of the invention have an X group present in the 4-position of the essential benzene ring, that X group does not represent halo.

In more particular embodiments, where compounds of the invention have an X group present in the 4-position of the essential benzene ring, that X group does not represent chloro.

For the avoidance of doubt, when R¹ represents n-butyl, R² and R³ represent H, n represents 1 and X is —OH and is in the 4-position on the phenyl group to which it is attached, the compound of formula I may be depicted as:

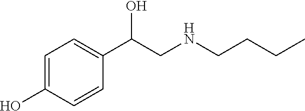

In further particular embodiments of the compounds of the invention, the compound of formula I is a compound of formula IA

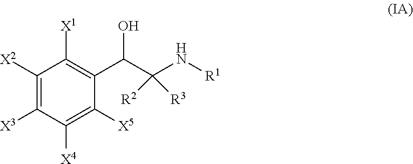

wherein R¹, R² and R³ are as defined herein (for the avoidance of doubt, including all embodiments thereof), and X¹, X², X³, X⁴ and X⁵ each independently represent H or X, wherein X is as defined herein (for the avoidance of doubt, including all embodiments thereof).

In certain embodiments, there is provided a compound of formula IA, wherein:

X¹ and X⁵ each independently represent H, fluoro, chloro or methyl; and

X², X³ and X⁴ each independently represent H, halo, R^{a}, —CN, —N₃, —N(R^{b})R^{c}, —NO₂ or —OR^{d}, wherein R^{a} represents C₁₋₄ alkyl optionally substituted by one or more F, and wherein R^{b}, R^{c} and R^{d} each independently represent H or C₁₋₄ alkyl optionally substituted by one or more F.

In particular embodiments, there is provided a compound of formula IA, wherein X¹ represents H.

In further embodiments, there is provided a compound of formula IA, wherein X³ represents H, —NH₂ or —OH.

In particular embodiments, there is provided a compound of formula IA, wherein X³ represents —NH₂ or —OH.

In more particular embodiments, there is provided a compound of formula IA, wherein X³ represents —OH.

Thus, in further embodiments, there is provided a compound of formula IA, wherein:

X¹ represents H; and

X⁵ represents H, fluoro, chloro or methyl; and

X², X³ and X⁴ each independently represent H, halo, R^{a}, —CN, —N₃, —N(R^{b})R_{c}, —NO₂ or —OR^{d}, wherein R^{a} represents C₁₋₄ alkyl optionally substituted by one or more F, and wherein R^{b}, R^{c} and R^{d} each independently represent H or C₁₋₄ alkyl optionally substituted by one or more F.

In particular embodiments:

X¹, X² and X⁵ each represent H; and

X³ and X⁴ each independently represent H, halo, R^{a}, —CN, —NH₂, or —OH, wherein R^{a} represents C₁₋₂ alkyl optionally substituted by one or more F (for example R^{a} may represent —CF₃ or —CHF₂).

In further particular embodiments:

X¹, X² and X⁵ each represent H; and

X³ and X⁴ each independently represent H, halo, —NH₂, —CN or —OH.

In more particular embodiments:

X¹, X² and X⁵ each represent H; and

X³ and X⁴ each independently represent H, halo (e.g. F, Cl), —CN or —OH.

In yet more particular embodiments:

X¹, X² and X⁵ each represent H; and

X³ and X⁴ each independently represent H, Cl or —OH.

In yet more particular embodiments:

X¹, X² and X⁵ each represent H;

X³ represent —NH₂ or —OH; and/or (e.g. and)

X⁴ represents H or Cl.

In certain embodiments:

X¹, X², X³, X⁴ and X⁵ each represent H; or

X¹, X², X³ and X⁵ represent H and X⁴ represents Cl; or

X¹, X², X⁴ and X⁵ represent H and X³ represents —OH.

In alternative embodiments:

X¹ and X⁵ each represent H.

X² and X⁴ each independently represent halo, R^{a}, —CN, —N₃, —N(R^{b})R^{c}, —NO₂ or —OR^{d};

wherein R^{a} represents C₁₋₄ alkyl optionally substituted by one or more F, and wherein R^{b}, R^{c} and R^{d} each independently represents H or C₁₋₄ alkyl optionally substituted by one or more F; and

X³ represents H, halo, R^{a}, —CN, —N₃, —N(R^{b})R^{c}, —NO₂ or —OR^{d}; wherein R^{a} represents C₁₋₄ alkyl optionally substituted by one or more F, and wherein R^{b}, R^{c} and R^{d} each independently represents H or C¹⁻⁴ alkyl optionally substituted by one or more F.

In further alternative embodiments:

X¹ and X⁵ each represent H;

X² and X⁴ each independently represent F, Cl, R^{a} or OR^{d}; wherein R^{a} represents C₁₋₂ alkyl optionally substituted by one or more F, and wherein R^{d} represents H or C₁₋₂ alkyl optionally substituted by one or more F; and

X³ represents H, —N(R^{b})R^{c} or —OR^{d}; wherein R^{b}, R^{c} and R^{d} each independently represent

H or C₁₋₂ alkyl optionally substituted by one or more F.

In yet more particular embodiments:

X¹ and X⁵ each represent H;

X² and X⁴ each independently represent F, Cl, —CF₃ or —OH; and

X³ represents H, —NH₂ or —OH.

In yet more alternative embodiments:

X¹ and X⁵ each represent H;

X² and X⁴ each independently represent chloro, —CF₃ or —OH; and

X³ represents H, —NH₂ or —OH.

In certain embodiments:

X¹, X³ and X⁵ each represent H; and

X² and X⁴ each represent —OH.

In further certain embodiments:

X¹ and X⁵ represent H

X² and X⁴ each independently represent Cl or —CF₃, and

X³ represents —NH₂ or —OH.

In certain instances, X¹ represents —Cl and X³ represents —CF₃.

The skilled person will understand that particular X groups (and the positions and number thereof, such as may correspond to X¹ to X⁵ groups in compounds of formula IA) that may be mentioned include those present in the examples provided herein.

Similarly, the skilled person will understand that particular R¹, R² and R³ groups that may be mentioned include those present in the examples provided herein.

For example, in a particular embodiment of the first aspect of the invention:

R¹ represents n-butyl or cyclopropylmethyl;

R² and R³ represent H;

n represents 1; and

X represents —OH and is in the 4-position on the phenyl group to which it is attached (i.e. in a compound of formula IA, X¹, X², X⁴ and X⁵ represent H and X³ represents —OH).

Particular compounds of the first aspect of the invention that may be mentioned include the compounds of the examples provided herein, and pharmaceutically acceptable salts thereof. Thus, compounds of formula I that may be mentioned include:

4-(2-(butylamino)-1-hydroxyethyl)phenol,

2-(butylamino)-1-(3-chlorophenyl)ethan-1-ol,

4-(2-((cyclopropylmethyl)amino)-1-hydroxyethyl)phenol,

4-(2-(tert-butylamino)-1-hydroxyethyl)phenol,

4-(2-(sec-butylamino)-1-hydroxyethyl)phenol,

4-(2-((cyclohexylmethyl)amino)-1-hydroxyethyl)phenol,

4-(2-(cyclopentylamino)-1-hydroxyethyl)phenol,

4-(1-hydroxy-2-(pentan-2-ylamino)ethyl)phenol,

4-(2-(adamantan-1-ylamino)-1-hydroxyethyl)phenol,

2-((cyclohexylmethyl)amino)-1-phenylethan-1-ol,

4-(2-(butylamino)-1-hydroxypropyl)phenol,

4-(1-hydroxy-2-(pentan-2-ylamino)propyl)phenol,

3-(2-(butylamino)-1-hydroxyethyl)phenol,

3-(1-hydroxy-2-(pentan-2-ylamino)ethyl)phenol,

4-(2-((2-cyclohexylethyl)amino)-1-hydroxyethyl)phenol,

4-(2-(hexylamino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

5-(2-(butylamino)-1-hydroxyethyl)benzene-1,3-diol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3,5-dichlorophenyl)-2-(butylamino)ethan-1-ol,

1-(4-amino-3-chloro-5-(trifluoromethyl)phenyI)-2-(butylamino)ethan-1-ol,

1-(4-amino-3-chloro-5-(trifluoromethyl)phenyl)-2-(pentan-2-ylamino)ethan-1-ol,

5-(2-(butylamino)-1-hydroxyethyl)-2-chlorophenol,

2-chloro-5-(1-hydroxy-2-(pentan-2-ylamino)ethyl)phenol,

1-(3-amino-4-chlorophenyl)-2-(butylamino)ethan-1-ol, and

1-(4-amino-3,5-difluorophenyl)-2-(butylamino)ethan-1-ol, and pharmaceutically acceptable salts thereof.

More particular compounds of formula I include:

4-(2-(butylamino)-1-hydroxyethyl)phenol,

2-(butylamino)-1-(3-chlorophenyl)ethan-1-ol,

4-(2-((cyclopropylmethyl)amino)-1-hydroxyethyl)phenol,

4-(2-(tert-butylamino)-1-hydroxyethyl)phenol,

4-(2-(sec-butylamino)-1-hydroxyethyl)phenol,

4-(2-((cyclohexylmethyl)amino)-1-hydroxyethyl)phenol,

4-(2-(cyclopentylamino)-1-hydroxyethyl)phenol,

4-(1-hydroxy-2-(pentan-2-ylamino)ethyl)phenol,

4-(2-(adamantan-1-ylamino)-1-hydroxyethyl)phenol,

2-((cyclohexylmethyl)amino)-1-phenylethan-1-ol,

4-(2-(butylamino)-1-hydroxypropyl)phenol,

4-(1-hydroxy-2-(pentan-2-ylamino)propyl)phenol,

3-(2-(butylamino)-1-hydroxyethyl)phenol,

3-(1-hydroxy-2-(pentan-2-ylamino)ethyl)phenol,

4-(2-((2-cyclohexylethyl)amino)-1-hydroxyethyl)phenol,

4-(2-(hexylamino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

5-(2-(butylamino)-1-hydroxyethyl)benzene-1,3-diol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3,5-dichlorophenyl)-2-(butylamino)ethan-1-ol,

1-(4-amino-3-chloro-5-(trifluoromethyl)phenyI)-2-(butylamino)ethan-1-ol, and

1-(4-amino-3-chloro-5-(trifluoromethyl)phenyI)-2-(pentan-2-ylamino)ethan-1-ol, and pharmaceutically acceptable salts thereof.

Certain particular compounds of formula I include:

4-(2-(butylamino)-1-hydroxyethyl)phenol,

2-(butylamino)-1-(3-chlorophenyl)ethan-1-ol,

4-(2-((cyclopropylmethyl)amino)-1-hydroxyethyl)phenol,

4-(2-(tert-butylamino)-1-hydroxyethyl)phenol,

4-(2-(sec-butylamino)-1-hydroxyethyl)phenol,

4-(2-((cyclohexylmethyl)amino)-1-hydroxyethyl)phenol,

4-(2-(cyclopentylamino)-1-hydroxyethyl)phenol,

4-(1-hydroxy-2-(pentan-2-ylamino)ethyl)phenol,

4-(2-(adamantan-1-ylamino)-1-hydroxyethyl)phenol, and

2-((cyclohexylmethyl)amino)-1-phenylethan-1-ol, and pharmaceutically acceptable salts thereof.

Further particular compounds of formula I that may be mentioned include:

4-(2-(butylamino)-1-hydroxyethyl)phenol,

4-(2-((cyclopropylmethyl)amino)-1-hydroxyethyl)phenol,

4-(2-(tert-butylamino)-1-hydroxyethyl)phenol,

4-(2-((cyclohexylmethyl)amino)-1-hydroxyethyl)phenol, and

2-((cyclohexylmethyl)amino)-1-phenylethan-1-ol, and pharmaceutically acceptable salts thereof.

Yet more particular compounds of formula I that may be mentioned include:

4-(2-(butylamino)-1-hydroxyethyl)phenol, and

4-(2-((cyclopropylmethyl)amino)-1-hydroxyethyl)phenol, and pharmaceutically acceptable salts thereof.

As described herein, compounds of the first aspect of the invention may also contain one or more asymmetric carbon atoms and may therefore exhibit optical and/or diastereoisomerism. Moreover, it has been found that certain such optical and/or diastereoisomers may show increased utility in the treatment of hyperglycaemia or disoders characterized by hyperglycaemia (such as type 2 diabetes), as described herein.

In a particular embodiment of the first aspect of the invention, the compound of formula I is such that the carbon substituted with the essential —OH group is in the R configuration, as understood by those skilled in the art.

Thus, in a particular embodiment, the compound of formula I is a compound of formula IB

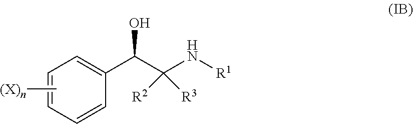

wherein n, X, R¹, R² and R³ are as described herein (i.e. as described in the first aspect of the invention, including all embodiments and particular features, and combinations thereof).

In particular embodiments, there is provided a compound of formula IB wherein:

n represents 1;

X represents —OH;

R¹ represents C₄₋₈ alkyl (e.g. C₄ alkyl, such as n-butyl); and/or (e.g. and)

R² and R³ both represent H.

In more particular embodiments, there is provided a compound of formula IB wherein:

n represents 1 and X represents —OH in the 4-position of the phenyl group;

R¹ represents C₄₋₈ alkyl (e.g. C₄ alkyl, such as n-butyl); and

R² and R³ both represent H.

In a yet more particular embodiment, the compound of formula I (or the compound of formula IA or IB) is a compound of formula IC

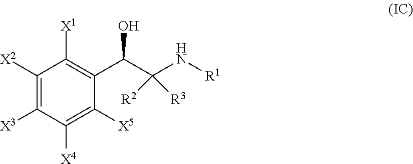

wherein X¹, X², X³, X⁴, X⁵, R¹, R² and R³ are as described herein (i.e. as described in the first aspect of the invention, including all embodiments and particular features, and combinations thereof).

For example, there is provided a compound of formula IC wherein:

X¹, X³, X⁴ and X⁵ each represent H;

X³ represents —OH;

R¹ represents C₄₋₈ alkyl (e.g. C₄ alkyl, such as n-butyl); and/or (e.g. and)

R² and R³ both represent H.

As described herein, particular compounds of the first aspect of the invention that may be mentioned include the compounds of the examples provided herein, and pharmaceutically acceptable salts thereof. Thus, compounds of formula IB or IC that may be mentioned include:

(R)-4-(2-(butylamino)-1-hydroxyethyl)phenol,

and pharmaceutically acceptable salts thereof.

The skilled person will understand that references to a specific steroisomer of a compound of formula I (e.g. in the case of compounds of formula I, where the carbon substituted by the essential —OH group is in the R configuration, as represented by compounds of formula IB and formula IC) will refer to the specific stereoisomer present in the substantial absence of the corresponding opposite stereoisomer (e.g. in the case of compounds of formula I, where the carbon substituted by the essential —OH group is in the S configuration).

For example, references to a compound of formula IC being present in the substantial absence of the corresponding opposite steroisomer (i.e. in the S configuration) will refer to the substantial absence of the corresponding compound as depicted below.

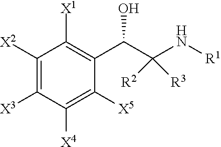

As used herein, references to the substantial absence of the corresponding opposite stereoisomer will refer to the desired stereoisomer (e.g. in the case of compounds of formula I, where the carbon substituted by the essential —OH group is in the (R) configuration) being present at a purity of at least 80% (e.g. at least 90%, such as at least 95%) relative to the opposite stereoisomer (e.g. in the case of compounds of formula I, where the carbon substituted by the essential —OH group is in the S configuration). Alternatively, in such instances, compounds may be indicated to be present in the substantial absence of the compound in the other configuration (i.e. (S) configuration), which may indicate that the compound in the relevant configuration is present in an enantiomeric excess (e.e.) of at least 90% (such as at least 95%, at least 98% or, particularly, at least 99%, for example at least 99.9%).

For the avoidance of doubt, compounds referred to as having a specific stereochemistry at a defined position (e.g. in the case of compounds of formula I, the carbon substituted by the essential —OH group being in the R configuration) may also have stereochemistry at one or more other positions, and so may exist as mixtures of enantiomers or diastereoisomers in relation to the stereochemistry at those positions.

For the avoidance of doubt, the term “hyperglycaemia” as used herein will be understood by those skilled in the art to refer to a condition wherein an excessive amount of glucose circulates in blood plasma of the subject experiencing the same. In particular, it may refer to a subject (e.g. a human subject) having blood glucose levels higher than about 10.0 mmol/L (such as higher than about 11.1 mmol/L, e.g. higher than about 15 mmol/L), although it may also refer to a subject (e.g. a human subject) having blood glucose levels higher than about 7 mmol/L for an extended period of time (e.g. for greater than 24 hours, such as for greater than 48 hours).

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of type 2 diabetes, the term may refer to achieving a reduction of blood glucose levels. In particular embodiments, in the case of treating hyperglycaemia or conditions characterised by hyperglycaemia, the term may refer to achieving a reduction of blood glucose levels (for example, to or below about 10.0 mmol/mL (e.g. to levels in the range of from about 4.0 mmol/L to about 10.0 mmol/L), such as to or below about 7.5 mmol/mL (e.g. to levels in the range of from about 4.0 mmol/L to about 7.5 mmol/L) or to or below about 6 mmol/mL (e.g. to levels in the range of from about 4.0 mmol/L to about 6.0 mmol/L)).

As used herein, references to patients will refer to a living subject being treated, including mammalian (e.g. human) patients. Thus, in particular embodiments of the first aspect of the invention, the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

Although compounds of the first aspect of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the first aspect of the invention are included within the scope of the invention.

For the avoidance of doubt, the compounds of the first aspect of the invention are useful because they possess pharmacological activity, and/or are metabolised in the body following oral or parenteral administration to form compounds that possess pharmacological activity. In particular, as described herein, compounds of the first aspect of the invention are useful in the treatment of hyperglycaemia or disorders characterized by hyperglycaemia (such as type 2 diabetes), which terms will be readily understood by one of skill in the art (as described herein).

In a particular embodiment, the treatment is of a disorder (which may also be referred to as a condition or disease) characterised by hyperglycaemia.

In particular embodiments, compounds of the invention (i.e. compounds of formula I, including all embodiments thereof) are for use in the treatment of type 2 diabetes (or useful in the manufacture of a medicament for such treatment, or useful in a method for such treatment, as described herein).

In particular embodiments of the first aspect of the invention, the disorder is type 2 diabetes, such as type 2 diabetes of a sub-type selected from the list consisting of maturity-onset diabetes in the young (MODY), ketosis-prone diabetes in adults, latent autoimmune diabetes of adults (LADA), and gestational diabetes.

In further particular embodiments, the treatment of type 2 diabetes is in a non-obese patient.

For the avoidance of doubt, the skilled person will understand that patients with a Body Mass Index (BMI) of greater than 30 are considered to be obese.

In particular embodiments, the treatment may be of hyperglycaemia in a patent who is at risk of developing type 2 diabetes, which condition may be defined as pre-diabetes. Thus, compounds of the invention may be useful in the prevention of type 2 diabetes (e.g. in a patient having pre-diabetes).

As used herein, the term prevention (and, similarly, preventing) includes references to the prophylaxis of the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In more particular embodiments, the type 2 diabetes is characterised by the patient displaying severe insulin resistance (SIR).

In further embodiments, the treatment may be of hyperglycaemia in a patient having type 1 diabetes. Thus, compounds of the invention may be useful in the treatment of hyperglycaemia in type 1 diabetes.

The skilled person will understand that compounds of the invention may be useful in treating hyperglycaemia in patients having impaired insulin production, such as in patients having cystic fibrosis. Thus, in further embodiments, the disorder characterized by hyperglycaemia is cystic fibrosis-related diabetes.

In particular embodiments that may be mentioned, the disorder characterised by hyperglycaemia is (or is characterized by) severe insulin resistance (SIR), which may be understood by those in the art to refer to disorders wherein typically the subject has normal, or in some cases increased, insulin production but significantly reduced insulin sensitivity. In particular instances, such patients may be non-obese (e.g. being of a healthy weight). Thus, in particular embodiments, such treatments are performed in patients who are not defined as being obese (e.g. in patients who are defined as being of a healthy weight).

For example, SIR may be identified in a patient based in said patient having fasting insulin >150 pmol/L and/or a peak insulin on glucose tolerance testing of >1,500 pmol/L, particularly in individuals with a BMI <30kg/m² (which patient may otherwise have normal glucose tolerance).

More particularly, SIR may be characterised by the patient having no significant response to the presence of insulin, which may result from a defect (e.g. a genetic defect) in the function of the insulin receptor.

Particular disorders that may be characterised by SIR include: Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

More particular disorders that may be characterised by SIR include Donohue's syndrome and Type A syndrome of insulin resistance and, yet more particularly, Rabson-Mendenhall syndrome.

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that:

(i) are capable of reducing blood sugar levels; and/or

(ii) are insulin sensitizers; and/or

(iii) enhance insulin release, all of which are described herein below.

Novel Compounds and Medical Uses

Certain compounds of the invention as disclosed herein (such as in the examples) may be novel and/or not previously disclosed for use in medicine.

In a second aspect of the invention, there is provided a compound selected from the group consisting of:

4-(2-((cyclopropylmethyl)amino)-1-hydroxyethyl)phenol,

4-(1-hydroxy-2-(pentan-2-ylamino)propyl)phenol,

3-(1-hydroxy-2-(pentan-2-ylamino)ethyl)phenol,

4-(2-((2-cyclohexylethyl)amino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3-chloro-5-(trifluoromethyl)phenyl)-2-(butylamino)ethan-1-ol,

1-(4-amino-3-chloro-5-(trifluoromethyl)phenyl)-2-(pentan-2-ylamino)ethan-1-ol,

5-(2-(butylamino)-1-hydroxyethyl)-2-chlorophenol,

2-chloro-5-(1-hydroxy-2-(pentan-2-ylamino)ethyl)phenol, and

1-(4-amino-3,5-difluorophenyI)-2-(butylamino)ethan-1-ol, and pharmaceutically acceptable salts thereof.

In a particular embodiment of the second aspect of the invention, the compound is selected from the group consisting of:

4-(2-((cyclopropylmethyl)amino)-1-hydroxyethyl)phenol,

4-(1-hydroxy-2-(pentan-2-ylamino)propyl)phenol,

3-(1-hydroxy-2-(pentan-2-ylamino)ethyl)phenol,

4-(2-((2-cyclohexylethyl)amino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3-chloro-5-(trifluoromethyl)phenyI)-2-(butylamino)ethan-1-ol, and

1-(4-amino-3-chloro-5-(trifluoromethyl)phenyl)-2-(pentan-2-ylamino)ethan-1-ol, and pharmaceutically acceptable salts thereof.

In a more particular embodiment of the second aspect of the invention, the compound is

4-(2-((cyclopropylmethyl)amino)-1-hydroxyethyl)phenol, or a pharmaceutically-acceptable salt thereof.

In a third aspect of the invention, there is provided a compound selected from the group consisting of:

2-(butylamino)-1-(3-chlorophenyl)ethan-1-ol,

4-(2-((cyclopropylmethyl)amino)-1-hydroxyethyl)phenol,

4-(2-((cyclohexylmethyl)amino)-1-hydroxyethyl)phenol,

4-(2-(adamantan-1-ylamino)-1-hydroxyethyl)phenol,

2-((cyclohexylmethyl)amino)-1-phenylethan-1-ol,

4-(2-(butylamino)-1-hydroxypropyl)phenol,

3-(1-hydroxy-2-(pentan-2-ylamino)ethyl)phenol,

3-(1-hydroxy-2-(pentan-2-ylamino)ethyl)phenol,

4-(2-((2-cyclohexylethyl)amino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3-chloro-5-(trifluoromethyl)phenyI)-2-(butylamino)ethan-1-ol, and

1-(4-amino-3-chloro-5-(trifluoromethyl)phenyI)-2-(pentan-2-ylamino)ethan-1-ol,

5-(2-(butylamino)-1-hydroxyethyl)-2-chlorophenol,

2-chloro-5-(1-hydroxy-2-(pentan-2-ylamino)ethyl)phenol,

1-(3-amino-4-chlorophenyl)-2-(butylamino)ethan-1-ol, and

1-(4-amino-3,5-difluorophenyl)-2-(butylamino)ethan-1-ol, and pharmaceutically acceptable salts thereof, for use in medicine (which may also be referred to as use as a pharmaceutical).

In particular embodiments of the third aspect of the invention, the compound is selected from the group consisting of:

2-(butylamino)-1-(3-chlorophenyl)ethan-1-ol,

4-(2-((cyclopropylmethyl)amino)-1-hydroxyethyl)phenol,

4-(2-((cyclohexylmethyl)amino)-1-hydroxyethyl)phenol,

4-(2-(adamantan-1-ylamino)-1-hydroxyethyl)phenol,

2-((cyclohexylmethyl)amino)-1-phenylethan-1-ol,

4-(2-(butylamino)-1-hydroxypropyl)phenol,

3-(1-hydroxy-2-(pentan-2-ylamino)ethyl)phenol,

3-(1-hydroxy-2-(pentan-2-ylamino)ethyl)phenol,

4-(2-((2-cyclohexylethyl)amino)-1-hydroxyethyl)phenol,

4-(1-hydroxy-2-(octylamino)ethyl)phenol,

2-chloro-4-(1-hydroxy-2-(pentan-2-ylamino)ethyl)phenol,

4-(2-(butylamino)-1-hydroxyethyl)-2-chlorophenol,

4-(2-((3-cyclohexylpropyl)amino)-1-hydroxyethyl)phenol,

4-(2-((2-cyclopropylethyl)amino)-1-hydroxyethyl)phenol,

4-(2-((3-cyclopropylpropyl)amino)-1-hydroxyethyl)phenol,

4-(1-hydroxy-2-((4,4,4-trifluorobutyl)amino)ethyl)phenol,

4-(2-(butylamino)-1-hydroxyethyl)-2,6-dichlorophenol,

2,6-dichloro-4-(1-hydroxy-2-(pentan-2-ylamino)ethyl)phenol,

1-(4-amino-3,5-dichlorophenyl)-2-(pentan-2-ylamino)ethan-1-ol,

1-(4-amino-3-chloro-5-(trifluoromethyl)phenyI)-2-(butylamino)ethan-1-ol, and

1-(4-amino-3-chloro-5-(trifluoromethyl)phenyI)-2-(pentan-2-ylamino)ethan-1-ol, and pharmaceutically acceptable salts thereof

In more particular embodiments of the third aspect of the invention, the compound is selected from the group consisting of:

2-(butylamino)-1-(3-chlorophenyl)ethan-1-ol,

4-(2-((cyclopropylmethyl)amino)-1-hydroxyethyl)phenol,

4-(2-((cyclohexylmethyl)amino)-1-hydroxyethyl)phenol,

4-(2-(adamantan-1-ylamino)-1-hydroxyethyl)phenol, and

2-((cyclohexylmethyl)amino)-1-phenylethan-1-ol, and pharmaceutically-acceptable salts thereof.

Pharmaceutical Compositions

As described herein, compounds of the first and, therefore, the second and third aspects of the invention are useful as pharmaceuticals. Such compounds may be administered alone or may be administered by way of known pharmaceutical compositions/formulations.

In a fourth aspect of the invention, there is provided a pharmaceutical composition comprising a compound as defined in the second or third aspect of the invention, and optionally one or more pharmaceutically acceptable adjuvant, diluent and/or carrier.

The skilled person will understand that references herein to compounds of the first aspect of the invention being for particular uses (and, similarly, to uses and methods of use relating to compounds of the invention) may also apply to pharmaceutical compositions comprising compounds of the invention as described herein.

In a fifth aspect of the invention, there is provided a pharmaceutical composition for use in the treatment of hyperglycaemia or a disoder characterized by hyperglycaemia (as defined herein, such as type 2 diabetes) comprising a compound as defined in the first aspect of the invention, and optionally one or more pharmaceutically acceptable adjuvant, diluent and/or carrier.

The skilled person will understand that compounds of the first (and, therefore, second and third) aspect of the invention may act systemically and/or locally (i.e. at a particular site).

The skilled person will understand that compounds and compositions as described in the first to fifth aspects of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments of the fourth and fifth aspects of the invention, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that compounds of the invention, and pharmaceutically-acceptable salts thereof, may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 10 mg, 20 mg, 30 mg or 40 mg twice daily, or 10 μg, 20 μg, 30 μg or 40 μg twice daily).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

As described herein above, the skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of hyperglycaemia or a disoder characterized by hyperglycaemia(as defined herein, such as type 2 diabetes), such as treatment with one or more other therapeutic agent that is useful in the treatment of hyperglycaemia or a disoder characterized by hyperglycaemia(as defined herein, such as type 2 diabetes).

In particular embodiments of the fourth and fifth aspects of the invention, the pharmaceutical composition may further comprise one or more additional (i.e. other) therapeutic agent.

In more particular embodiments, the one or more additional therapeutic agent is an agent for the treatment of type 2 diabetes as known to those skilled in the art, such as metformin, sulfonylureas (e.g. carbutamide, acetohexamide, chlorpropamide, tolbutamide. glipizide (glucotrol), gliclazide, glibenclamide, glyburide (Micronase), glibornuride, gliquidone, glisoxepide, glyclopyramide, glimepiride (Amaryl), glimiprime, JB253 or JB558), thiazolidinediones (e.g. pioglitazone, rosiglitazone (Avandia), lobeglitazone (Duvie) and troglitazone (Rezulin)), dipeptidyl peptidase-4 inhibitors (e.g. sitagliptin, vildagliptin, saxagliptin, linagliptin, anagliptin, teneligliptin, alogliptin, trelagliptin, gemigliptin, dutogliptin and omarigliptin), SGLT2 inhibitors (e.g. dapagliflozin, empagliflozin, canagliflozin, ipragliflozin, tofogliflozin, sergliflozin etabonate, remogliflozin etabonate, and ertugliflozin), and glucagon-like peptide-1 (GLP-1) analogues.

The skilled person will understand that combinations of therapeutic agents may also described as a combination product and/or provided as a kit-of-parts.

In a sixth aspect of the invention, there is provided a combination product comprising:

(A) a compound as defined in the first aspect of the invention; and

(B) one or more additional therapeutic agent, wherein each of components (A) and (B) is formulated in admixture, optionally with one or more a pharmaceutically-acceptable adjuvant, diluent or carrier.

In a seventh aspect of the invention, there is provided a kit-of-parts comprising:

(a) a compound as defined in the first (or second and/or third) aspect of the invention, (or a pharmaceutical composition comprising the same) or a pharmaceutical composition as defined in the fourth or fifth aspect of the invention; and

(b) one or more other therapeutic agent, optionally in admixture with one or more pharmaceutically-acceptable adjuvant, diluent or carrier,

which components (a) and (b) are each provided in a form that is suitable for administration in conjunction with the other.

In particular embodiments (e.g. of the sixth and seventh aspects of the invention), the additional therapeutic agent is a therapeutic agent that is useful for the treatment of hyperglycaemia or a disorder characterized by hyperglycaemia (e.g. type 2 diabetes), as known to those skilled in the art (such as those described herein).

For example, in particular embodiments of the fourth to fifth aspects of the invention, the additional therapeutic agent is an agent that:

(i) is capable of reducing blood sugar levels; and/or

(ii) is an insulin sensitizer; and/or

(iii) is able to enhance insulin release,

which agents will be readily identified by those skilled in the art and include, in particular, such therapeutic agents that are commercially available (e.g. agents that the subject of a marketing authorization in one or more territory, such as a European or US marketing authorization).

The skilled person will understand that references to therapeutic agents capable of reducing blood glucose levels may refer to compounds capable of reducing levels of blood by at least 10% (such as at least 20%, at least 30% or at least 40%, for example at least 50%, at least 60%, at least 70% or at least 80%, e.g. at least 90%) when compared to the blood glucose levels prior to treatment with the relevant compound.

Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable adjuvant, diluent or carrier.

In further aspects of the invention, there is provided a process for the preparation of a combination product or kit-of-parts as hereinbefore defined, which process comprises bringing into association a compound of the invention, as hereinbefore defined, or a pharmaceutically acceptable salt thereof with the other therapeutic agent that is useful in the treatment of hyperglycaemia or a disorder characterized by hyperglycaemia (e.g. type 2 diabetes), and at least one pharmaceutically-acceptable adjuvant, diluent or carrier.

As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Compounds as defined in the first (and, therefore, second and third) aspect of the invention may be prepared in accordance with techniques that are well known to those skilled in the art, such as those described in the examples provided hereinafter.

For example, there is provided a process for the preparation of a compound of formula I, or a pharmaceutically acceptable salt thereof, as defined in the first aspect of the invention (which may be utilised in the preparation of, for example, a compound as defined in the second aspect of the invention), which process comprises:

(i) reaction of a compound of formula II

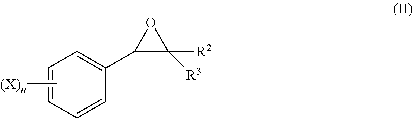

wherein n, X, R², R³ and n are as defined hereinabove, with a compound of formula III

H₂N—R¹ (III)

wherein R¹ is as defined hereinabove, optionally in the presence of a suitable solvent known to those skilled in the art;

(iia) reaction of a compound of formula IV

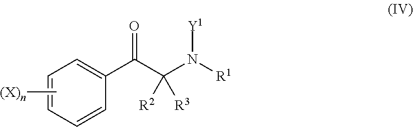

wherein n, X, R¹, R², R³ and n are as defined hereinabove and Y¹ represents H or PG¹ wherein PG¹ is a suitable protecting group as known to those skilled in the art (e.g. —C(O)OtBu or —SO₂CH₃) with a suitable reduction agent as known to those skilled in the art, such as NaBH₄ or LiAlH₄, or by hydrogenation in the presence of a suitable catalyst;

(iib) for compounds of formula IB (and, similarly, compounds of formula IC) reaction of a compound of formula IV as defined herein above but wherein Y¹ represents PG¹ wherein PG¹ is a suitable protecting group as known to those skilled in the art (e.g. —C(O)OtBu) in the presence of a suitable catalyst (such as a complex between (1S, 2S)—(+)—N—(4-toluenesulphonyl)-1,2-diphenylethylene diamine and [Ru(cymene)Cl₂]₂)) in the presence of hydrogen or a suitable hydrogen donor (such as formic acid) and optionally in the presence of a base (e.g. Et₃N) and in the presence of a suitable solvent (such as CH₂Cl₂);

(iii) for compounds wherein at least one X is present and represents —OH, deprotection of a compound of formula V

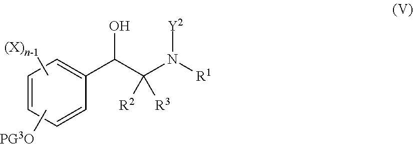

wherein n, R¹, R² and R³ are as defined hereinabove, Y² represents H or PG², wherein PG² represents a suitable protecting group as known to those skilled in the art, and PG³ represents a suitable protecting group as known to those skilled in the art (e.g. benzyl or alkyl, such as methyl) under conditions known to those skilled in the art (for example: in the case of benzyl, in the presence of hydrogen and a suitable catalyst or a suitable acid; in the case of alkyl, such as methyl, in the presence of BBr₃, HBr or alkyl sulfides).

(iv) for compounds wherein at least one X is present and represents NH₂, deprotection of a compound of formula VI

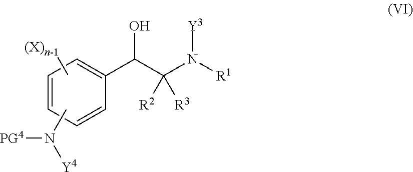

wherein n, X, R¹, R² and R³ are as defined hereinabove, Y³ represents H or PG⁵, wherein PG⁵ represents a suitable protecting group as known to those skilled in the art, Y⁴ represents H or PG⁶, wherein PG⁶ represents a suitable protecting group as known to those skilled in the art, and PG⁴ represents a suitable protecting group as known to those skilled in the art (e.g. carbamate protecting groups (such as tert-butyloxycarbonyl (Boc), fluorenylmethyloxycarbonyl (Fmoc) and carboxybenzyl (Cbz) and amide protecting groups (such as acetyl and benzoyl)) under conditions known to those skilled in the art (for example in the case of Boc, in the presence of a suitable add (e.g. trifluoroacetic add or HCl). PG⁴, PG⁵ (if present) and PG⁶ (if present) may each represent the same protecting group, and therefore may be deprotected under a single set of conditions;

(vii) for compounds wherein at least one X is present and represents NH₂, reduction of a compound of formula VII

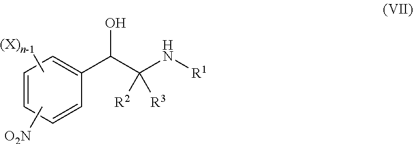

wherein n, X, R¹, R² and R³ are as defined hereinabove, under conditions known to those skilled in the art (for example, by hydrogenation, such as hydrogenation using hydrogen gas and a suitable catalyst as known to those skilled in the art,(e.g. Pd—C, PtO₂, Raney-Nickel), Fe or Zn in acidic media (e.g. AcOH), borohydrides together with a suitable catalyst (e.g. NaBH₄ and Raney-Nickel), or agents such as SnCl₂, TiCl₃, Sml₂, and the like. Those skilled in the art will understand that certain functional groups, such as the essential —OH and/or the —NHR¹ groups) may need to be protected (and deprotected) one or more times during the reaction, which protections (and deprotections) may be performed using techniques known to those skilled in the art.

Compounds of formulae II, III, IV, V, VI and VII are either commercially available, are known in the literature, or may be obtained either by analogy with the processes described herein, or by conventional synthetic procedures, in accordance with standard techniques, from available starting materials (e.g. appropriately substituted benzaldehydes, styrenes or phenacyl bromides (or phenacylchloride, and the like) using appropriate reagents and reaction conditions. In this respect, the skilled person may refer to inter alia “Comprehensive Organic Synthesis” by B. M. Trost and I. Fleming, Pergamon Press, 1991. Further references that may be employed include “Science of Synthesis”, Volumes 9-17 (Hetarenes and Related Ring Systems), Georg Thieme Verlag, 2006.

The substituents X, R¹, R² and R³, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Such compounds may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I or la).

The skilled person will understand that compounds of formula I having specific stereochemistry (such as compounds of formula IB and IC) may be provided by reacting suitable starting materials having the required stereochemistry in processes as described herein.

For example, compounds of formula IB and IC may be provided by reacting compounds having the required stereochemistry in processes as described in step (i) or step (iii) in the processes described herein above.

Further, the skilled person will understand that suitable starting materials having the required stereochemistry (such as suitable compounds of formula II and V wherein the carbon substituted with the essential oxygen is the R configuration, as required for the preparation of compounds of formula IB and IC) may be prepared by analogy with the process described in step (iib) herein above.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P.G.M. Wutz, Wiley-Interscience (1999).

Compounds as described herein (in particular, compounds as defined in the first and, therefore, second and third aspects of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, such compounds may have the advantage that they are more efficacious and/or exhibit advantageous properties in vivo.

Without wishing to be bound by theory, compounds as described herein are thought to be potent agonists of the β₂-adrenergic receptor, which allows for increased glucose uptake in skeletal muscle cells.

In addition, compounds as described herein are thought to be agonists of the β₂-adrenergic receptor without (or with only a minimal effect in) inducing cAMP production. It is thought that this allows for the increased glucose uptake in skeletal muscle cells with lower levels of side effects than would result from other treatments. Further, combining compounds as described herein with therapeutic agents that are able to decrease blood glucose levels is thought to provide an effective combination therapy.

### EXAMPLES

The present invention is illustrated by way of the following examples.

Chemicals and reagents were obtained from commercial suppliers and were used as received unless otherwise stated. All reactions involving moisture sensitive reagents were performed in oven or flame dried glassware under a positive pressure of nitrogen or argon.

Abbreviations

Abbreviations as used herein will be known to those skilled in the art. In particular, the following abbreviations may be used herein.

AcOH acetic acid

aq aqueous

atm atmosphere

Boc₂O di-tert-butyldicarbonate

DIPEA N,N-diisopropylethylamine

DMAP 4-dimethylaminopyridine

DMF dimethylformamide

DMSO dimethylsulfoxide

eq equivalent

EtOAc ethyl acetate

HPLC high-performance liquid chromatography

MeCN acetonitrile

MeOH methanol

Pd—C palladium on carbon

rt room temperature

sat saturated

TFA trifluoroacetic acid

THF tetrahydrofuran

### Example Compounds

In the event that there is a discrepancy between nomenclature and the structure of compounds as depicted graphically, it is the latter that presides (unless contradicted by any experimental details that may be given and/or unless it is clear from the context).

### Example 1

### 4-(2-(Butylamino)-1-hydroxyethyl)phenol

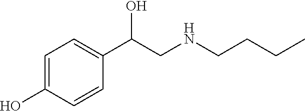

(a) 4-Methoxyphenyloxirane

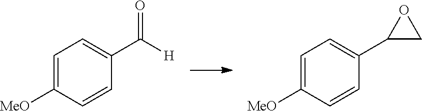 [SMILES: COc1ccc(C=O)cc1 ; COc1ccc(C2CO2)cc1]

A solution of trimethylsulfonium iodide (2.58 g, 12.7 mmol) in DMSO (20 mL) was added dropwise to an ice-cooled suspension of NaH (317 mg, 13.2 mmol, prepared from 529 mg 60% NaH in mineral oil) in THF (20 mL). To the ice-cooled mixture, a solution of 4-methoxybenzaldehyde (1.50 g, 11.0 mmol) in THF (7 mL) was slowly added. The cooling bath was removed, the mixture was stirred at rt for 20 h and poured onto ice. The mixture was extracted with Et₂O and the combined extracts were washed with water, brine, dried (Na₂SO₄) and concentrated to give the sub-title compound (1.61 g, 10.7 mmol, 97%) which was used in the next step without any further purification.

(b) 2-(Butylamino)-1-(4-methoxyphenyl)ethan-1-ol

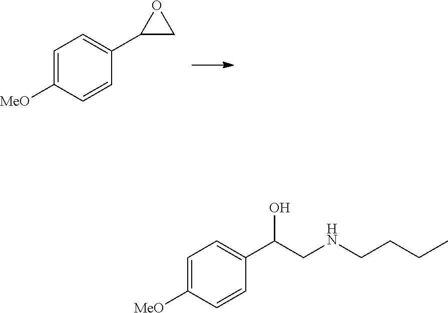 [SMILES: COc1ccc(C2CO2)cc1 ; CCCCNCC(O)c1ccc(OC)cc1]

A mixture of 4-methoxyphenyloxirane (500 mg, 3.33 mmol) and n-butylamine (1.3 mL, 13.3 mmol) was heated in a closed vial at 80° C. for 16 h. The mixture was concentrated and the residue crystallized from Et₂O/pentane (1:2) to give the sub-title compound (420 mg, 1.88 mmol, 56%).

(c) 4-(2-(Butylamino)-1-hydroxyethyl)phenol

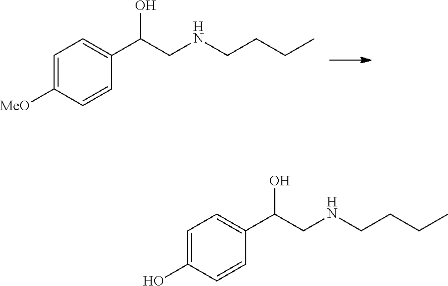 [SMILES: CCCCNCC(O)c1ccc(OC)cc1 ; CCCCNCC(O)c1ccc(O)cc1]

BBr₃ (0.22 mL, 2.2 mmol) was added to a solution of 2-(butylamino)-1-(4-methoxy-phenyl)ethan-1-ol (200 mg, 0.90 mmol) in 10 mL CH₂Cl₂ at 0° C. The cooling bath was removed and the mixture was stirred for 1 h and poured onto ice. The mixture was extracted with CH₂Cl₂ and the pH of the aqueous layer was adjusted to 6-7 with NaHCO₃ (aq, sat), extracted with CH₂Cl₂ and concentrated. The residue was suspended in EtOAc, dried (Na₂SO₄) and filtered trough Celite and concentrated. The residue was dissolved in EtOH (2 mL) and Et₂O (10 mL) was added. After standing in the freezer the precipitate was collected and the filtrate was concentrated and dissolved in EtOH (0.5 mL) and Et₂O (10 mL) was added. This second precipitate was collected, combined with the first and dried over P₂O₅ in vacuo to give the title compound (166 mg, 0.79 mmol, 89%). ¹H-NMR (400 MHz, THF-d⁸, drop of TFA): δ7.15-7.10 (m, 2H), 6.68-6.62 (m, 2H), 4.52 (dd, J=8.8, 4.0 Hz, 1H), 4.19 (br s, 1H), 2.66 (dd, J=11.7, 4.0 Hz, 1H), 2.63-2.52 (m, 3H), 1.48-1.28 (m, 4H), 0.90 (t, J=7.2 Hz, 3H).

### Example 2

### 2-(Butylamino)-1-(3-chlorophenyl)ethan-1-ol

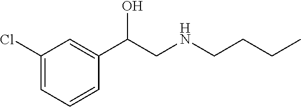

The title compound was prepared from 3-chlorophenyloxirane and butylamine in accordance with the preparation of Example 1.

¹H NMR (400 MHz, CDCl₃): δ7.38 (t, J=1.8 Hz, 1H), 7.30-7.26 (m, 1H), 7.26-7.21 (m, 2H), 4.66 (dd, J=9.1, 3.6 Hz, 1H), 2.90 (dd, J=12.2, 3.7 Hz, 1H), 2.73-2.54 (m, 4H), 1.52-1.43 (m, 2H), 1.41-1.30 (m, 2H), 0.92 (t, J=7.3 Hz, 3H).

### Example 3

### 4-(2-((Cyclopropylmethyl)amino)-1-hydroxyethyl)phenol

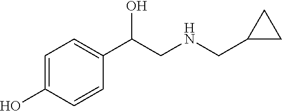 [SMILES: Oc1ccc(C(O)CNCC2CC2)cc1]

(a) 4-Benzyloxyacetophenone

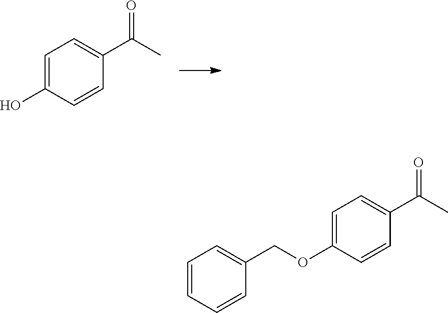

K₂CO₃ (7.5 g, 54.4 mmol) was added to a solution of 4-hydroxyacetophenone (3.7 g, 27.2 mmol) in acetone (180 mL) at rt. The mixture was stirred at rt for 1 h and benzylbromide (3.2 mL, 27.2 mmol) was added. The mixture was heated at reflux for 12 h, allowed to cool and filtered. Concentration of the filtrate gave the sub-title compound (6.1 g, 27.1 mmol, ˜100%) which was used in the next step without further purification.

(b) 4-Benzyloxyphenacyl bromide

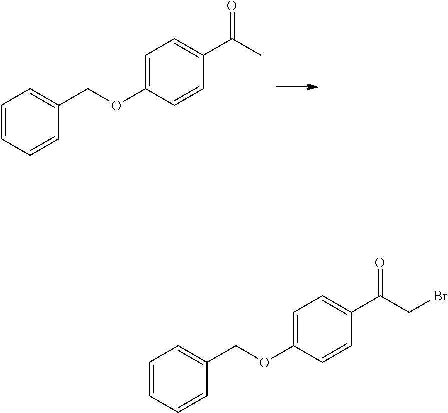 [SMILES: CC(=O)c1ccc(OCc2ccccc2)cc1 ; O=C(CBr)c1ccc(OCc2ccccc2)cc1]

Pyridinium tribromide (9.5 g, 29.8 mmol) was added to a solution of 4-benzyloxy-acetophenone (6.1 g, 27.1 mmol) in CH₂Cl₂ (275 mL) and MeOH (100 mL) at rt. After 3 h at rt the mixture was concentrated and the residue partitioned between water and EtOAc. The layers were separated and the aqueous layer was extracted with EtOAc. The combined organic phases were washed with brine, dried over Na₂SO₄ and filtered. Concentration of the filtrate and crystallization of the residue from hexane/EtOAc gave the sub-title compound (6.4 g, 21.1 mmol, 78%).

(c) 1-(4-Benzyloxyphenyl)-2-((cyclopropylmethyl)amino)ethan-1-ol

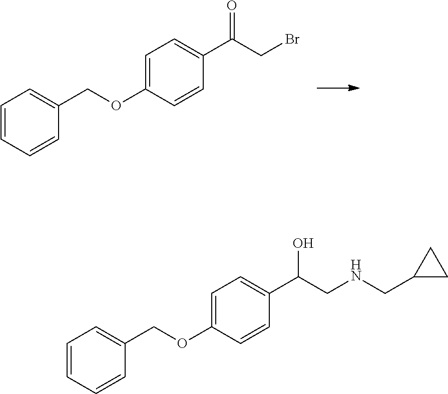 [SMILES: O=C(CBr)c1ccc(OCc2ccccc2)cc1 ; OC(CNCC1CC1)c1ccc(OCc2ccccc2)cc1]

A solution of 4-benzyloxyphenacyl bromide (250 mg, 0.82 mmol) in CH₂Cl₂ (5 mL) was added to a solution of cyclopropylmethylamine (0.28 mL, 3.3 mmol) in CH₂Cl₂ (5 mL) at −30° C. The mixture was stirred at −30° C. for 1 h and kept at −20° C. for 15 h. EtOH (10 mL) was added followed by NaBH₄ (93 mg, 2.5 mmol) in portions at 0° C. The mixture was stirred at rt for 16 h and cooled to 0° C. NH₄Cl was added until the gas evaluation ceased and the organic solvents were removed in vacuo. The mixture was extracted with EtOAc and the combined extracts were dried over Na₂SO₄ and concentrated. The residue was purified by chromatography to give the sub-title compound (122 mg, 0.41 mmol, 50%).

(d) 4-(2-((Cyclopropylmethyl)amino)-1-hydroxyethyl)phenol

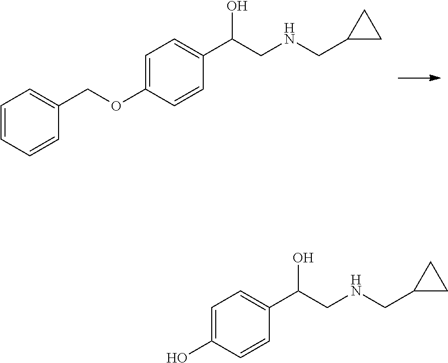 [SMILES: OC(CNCC1CC1)c1ccc(OCc2ccccc2)cc1 ; Oc1ccc(C(O)CNCC2CC2)cc1]

A mixture of 1-(4-benzyloxyphenyl)-2-((cyclopropylmethyl)amino)ethan-1-ol (100 mg, 0.34 mmol), 10% Pd—C (36 mg, 0.034 mmol) and EtOH (10 mL) was hydrogenated at 10 atm at rt for 2 h, filtered and concentrated. The filtrate was dissolved in hot EtOH (4 mL) and filtered. Et₂O (5 mL) was added, the mixture was allowed to cool and then kept in the fridge overnight. The precipitate was collected and dried to give the title compound (35 mg, 0.17 mmol, 50%).

¹H NMR (400 MHz, THF-d₈; a drop of TFA): δ9.85 (br s, 1H), 8.37 (br s, 1H), 7.24-7.15 (m, 2H), 6.75-6.66 (m, 2H), 4.99 (dd, J=10.7, 2.9 Hz, 1H), 3.27-3.19 (m, 1H), 3.08-2.97 (m, 2H), 2.97-2.87 (m, 1H), 1.23-1.12 (m, 1H), 0.65-0.60 (m, 2H), 0.42-0.37 (m, 2H).

### Example 4

### 4-(2-(tert-Butylamino)-1-hydroxyethyl)phenol

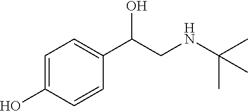

The title compound was prepared in accordance with the procedure in Example 3, Steps (c) and (d) from 4-benzyloxyphenacyl bromide an tert-butylamine.

¹H NMR (400 MHz, DMSO-d₆): δ9.22 (s, 1H), 7.16-7.06 (m, 2H), 6.72-6.65 (m, 2H), 4.99 (s, 1H), 4.38 (d, J=6.4 Hz, 1H), 2.53 (d, J=2.4 Hz, 1H), 2.52 (s, 1H), 1.00 (s, 9H).

### Example 5

### 4-(2-(sec-Butylamino)-1-hydroxyethyl)phenol

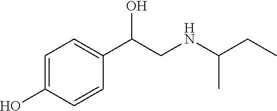

The title compound was prepared in accordance with the procedure in Example 3, Steps (c) and (d) from 4-benzyloxyphenacyl bromide and sec-butylamine.

¹H NMR (300 MHz, THF-d₈; a drop of TFA): δ9.91 (br s, 1H), 8.20 (br s, 1H), 7.06-7.39 (m, 2H), 6.84-6.55 (m, 2H), 5.13 (d, J=9.9 Hz, 1H), 3.32-3.07 (m, 2H), 3.05-2.84 (m, 1H), 2.08-1.86 (m, 1H), 1.67-1.53 (m, 1H), 1.36 (t, J=6.6 Hz, 3H), 0.98 (t, J=7.4 Hz, 3H).

### Example 6

### 4-(2-((Cyclohexylmethyl)amino)-1-hydroxyethyl)phenol

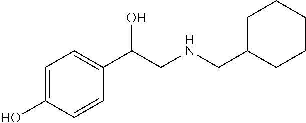 [SMILES: Oc1ccc(C(O)CNCC2CCCCC2)cc1]

The title compound was prepared in accordance with the procedure in Example 3, Steps (c) and (d) from 4-benzyloxyphenacyl bromide and cyclohexylmethylamine.

¹H NMR (300 MHz, THF-d₈; a drop of TFA): δ9.41 (br s, 1H), 8.17 (br s, 1H), 7.29-7.12 (m, 2H), 6.76-6.63 (m, 2H), 5.13 (dd, J=10.6, 2.8 Hz, 1H), 3.26-3.09 (m, 1H), 3.05-2.81 (m, 3H), 2.02-1.57 (m, 5H, overlapping with THF), 1.40-1.15 (m, 4H), 1.08-0.91 (m, 2H).

### Example 7

### 4-(2-(cyclopentylamino)-1-hydroxyethyl)phenol acetate

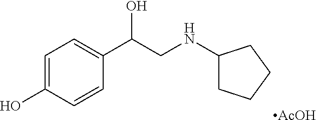 [SMILES: "·AcOH" ; Oc1ccc(C(O)CNC2CCCC2)cc1]

(a) 1-(4-(benzyloxy)phenyl)-2-(cyclopentylamino)ethan-1-ol

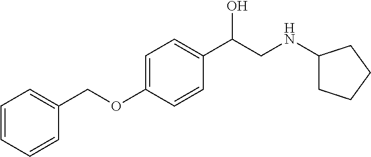 [SMILES: OC(CNC1CCCC1)c1ccc(OCc2ccccc2)cc1]

The sub-title compound was prepared in accordance with the procedure in Example 3, steps (c) and (d) from 4-benzyloxyphenacyl bromide and cyclopentylamine.

(b) 4-(2-(cyclopentylamino)-1-hydroxyethyl)phenol acetate

A mixture of 1-(4-(benzyloxy)phenyl)-2-(cyclopentylamino)ethan-1-ol (91 mg, 0.29 mmol), 10% Pd—C (31 mg, 0.029 mmol) and AcOH (8 mL) was hydrogenated at 10 atm at rt for 1 h, filtered through Celite and concentrated. The residue was treated with EtOH, refiltered through Celite and concentrated. The residue was repeatadly treated with Et₂O and concentrated to remove excess AcOH and finally dried in vacuo over KOH to give the title compound (79 mg, 0.28 mmol, 96%).

¹H NMR (300 MHz, D₂O): δ7.24-7.12 (m, 2H), 6.84-6.75 (m, 2H), 4.80 (dd, J=8.0, 5.2 Hz, 1H), 3.49 (quint, J=7.4 Hz, 1H), 3.19-3.06 (m, 2H), 2.05-1.88 (m, 2H), 1.77 (s, 3H), 1.69-1.41 (m, 6H).

### Example 8

### 4-(1-Hydroxy-2-(pentan-2-ylamino)ethyl)phenol acetate

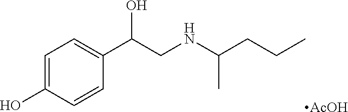 [SMILES: "·AcOH" ; CCCC(C)NCC(O)c1ccc(O)cc1]

The title compound was prepared in accordance with the procedure in Example 7, from 4-benzyloxyphenacyl bromide and 2-pentylamine.

¹H NMR (300 MHz, D₂O): δ7.44-7.25 (m, 2H), 7.05-6.84 (m, 2H), 5.02-4.90 (m, 1H), 3.50-3.17 (m, 3H), 1.92 (s, 3H), 1.81-1.26 (m, 4H), 1.33 (d, 3H), 0.93 (t, J=7.2 Hz, 3H).

### Example 9

### 4-(2-(adamantan-1-ylamino)-1-hydroxyethyl)phenol hemisulfate

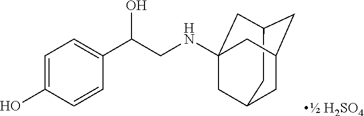

(a) 2-(adamantan-1-ylamino)-1-(4-(benzyloxy)phenyl)ethan-1-ol

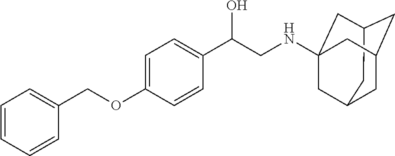 [SMILES: OC(CNC12CC3CC(CC(C3)C1)C2)c1ccc(OCc2ccccc2)cc1]

The sub-title compound was prepared in accordance with the procedure in Example 3, Steps (c) and (d) from 4-benzyloxyphenacyl bromide and 1-adamantylamine.

(b) 4-(2-(cyclopentylamino)-1-hydroxyethyl)phenol hemisulfate

A mixture of 2-(adamantan-1-ylamino)-1-(4-(benzyloxy)phenyl)ethan-1-ol (64 mg, 0.17 mmol), 10% Pd—C (31 mg, 0.017 mmol) and AcOH (7 mL) was hydrogenated at 10 atm at rt for 2 h, filtered through Celite and concentrated. The residue was treated with EtOH, refiltered through Celite and concentrated. The residue triturated with MeCN and treated with H₂O and 0.5 M H₂SO₄ (0.17 mL, 0.085 mmol). The solids where collected, washed with H₂O and dried to give the title compound (26 mg, 0.077 mmol, 46%).

¹H NMR (300 MHz, D₂O): δ7.41-7.27 (m, 2H), 7.03-6.85 (m, 2H), 4.90 (dd, J=8.9, 4.2 Hz, 1H), 3.37-3.12 (m, 2H), 2.21 (s, 3H), 2.02-1.85 (m, 6H), 1.71 (q, J=12.9 Hz, 6H).

### Example 10

### 2-((cyclohexylmethyl)amino)-1-phenylethan-1-ol

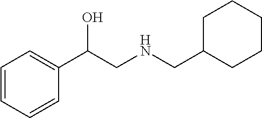 [SMILES: OC(CNCC1CCCCC1)c1ccccc1]

The title compound was purchased from Vitas M-Laboratory

### Example 11

### (R)-4-(2-(Butylamino)-1-hydroxyethyl)phenol hemisulfate

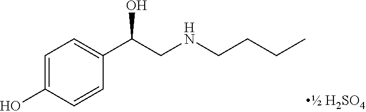

(a) tent-Butyl (2-(4-benzyloxyphenyl)-2-oxoethyl)butylcarbamate

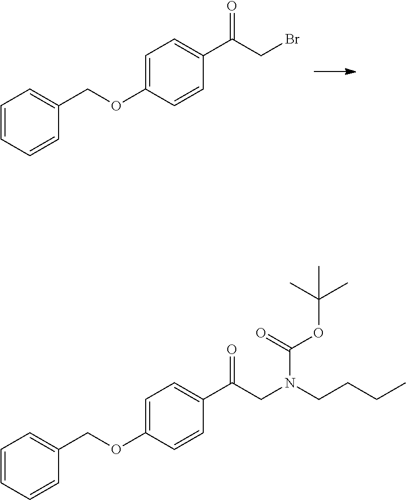 [SMILES: O=C(CBr)c1ccc(OCc2ccccc2)cc1 ; CCCCN(CC(=O)c1ccc(OCc2ccccc2)cc1)C(=O)OC(C)(C)C]

A solution of 4-benzyloxyphenacyl bromide (3.0 g, 10 mmol, see Example 3, step b) in CH₂Cl₂ (75 mL) was added to a solution of n-butylamine (3 mL, 30 mmol) in CH₂Cl₂ (75 mL) at −30° C. The mixture was stirred at −30° C. for 1 h and kept at −20° C. for 15 h. Di-tent-butyl dicarbonate (3.4 mL, 15 mmol) was added and the mixture was stirred at rt for 2 h. An additional portion of di-tent-butyl dicarbonate (2.3 mL, 10 mmol) was added and the mixture was stirred at rt for 2 h, washed with water and brine and dried over Na₂SO₄ and filtered. The filtrate was concentrated and purified by chromatography to give the sub-title compound (2.5 g, 6.3 mmol, 63%).

(b) tent-Butyl (R)-(2-(4-benzyloxyphenyI)-2-hydroxyethyl)butylcarbamate

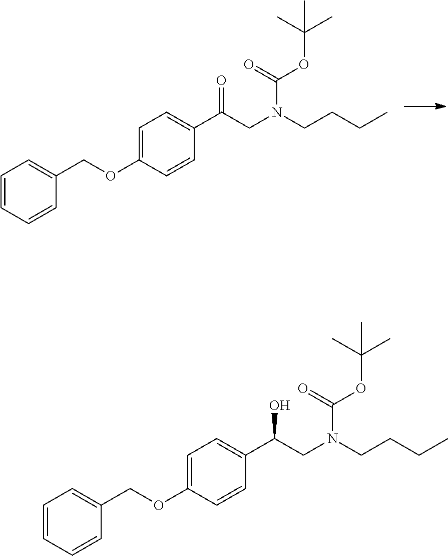 [SMILES: CCCCN(CC(=O)c1ccc(OCc2ccccc2)cc1)C(=O)OC(C)(C)C ; CCCCN(C[C@H](O)c1ccc(OCc2ccccc2)cc1)C(=O)OC(C)(C)C]

A transfer hydrogenation catalyst was prepared by dissolving (1S, 2S)—(+)—N—(4-toluenesulphonyl)-1,2-diphenylethylene diamine (36.9 mg, 0.10 mmol) and [Ru(cymene)Cl₂]₂ (30.8 mg, 0.05 mmol) in formic acid/Et₃N (5:2, 2 mL), as described in Kawamato, A. M. and Wills, M., J. Chem. Soc. Perkin 1, 1916 (2001). This solution was added to a mixture of tent-butyl (2-(4-benzyloxyphenyl)-2-oxoethyl)butylcarbamate (2 g, 5 mmol), formic acid/Et₃N (5:2, 7 mL) and CH₂Cl₂ (6 mL) at rt. The mixture was stirred for 60 h at rt and another portion of the catalyst/formic acid/Et₃N (same amounts as before, as prepared above) was added. The mixture was stirred at rt for 10 days, poured onto ice and extracted with CH₂Cl₂. The combined extracts were washed with brine, dried over Na₂SO₄ and concentrated. The residue was purified by chromatography to give the sub-title compound (1.9 g, 4.7 mmol, 94%).

(e) (R)-1-(4-Benzyloxyphenyl)-2-(butylamino)ethan-1-ol

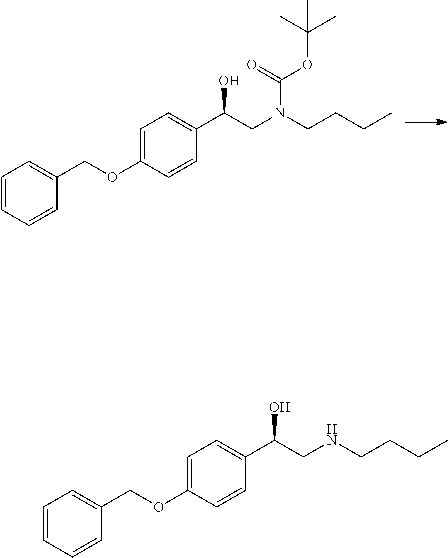 [SMILES: CCCCN(C[C@H](O)c1ccc(OCc2ccccc2)cc1)C(=O)OC(C)(C)C ; CCCCNC[C@H](O)c1ccc(OCc2ccccc2)cc1]

A solution of NaOH (3.6 g, 90 mmol) in water (30 mL) was added to a solution of tert-butyl (R)-(2-(4-benzyloxyphenyl)-2-hydroxyethyl)butylcarbamate (1.80 g, 4.5 mmol) in MeOH (60 mL). The mixture was heated at 100° C. in a sealed tube for 24 h, cooled and concentrated. Water was added and the precipitate was collected, washed with water and dried to give the sub-title compound (1.24 g, 4.2 mmol, 92%).

(f) (R)-4-(2-(Butylamino)-1-hydroxyethyl)phenol hemisulfate

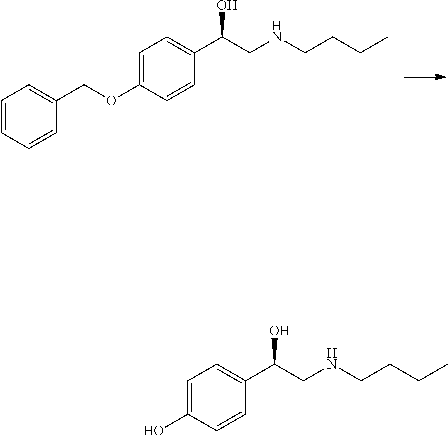 [SMILES: CCCCNC[C@H](O)c1ccc(OCc2ccccc2)cc1 ; CCCCNC[C@H](O)c1ccc(O)cc1]

A mixture of (R)-1-(4-benzyloxyphenyl)-2-(butylamino)ethan-1-ol (1.10 g, 3.67 mmol), 10% Pd—C (390 mg, 0.37 mmol) and AcOH (12 mL) was hydrogenated at 9 atm at rt for 2 h, filtered through Celite and concentrated. The residue was sonicated twice with MeCN, the MeCN was decanted off, and the residue was triturated with Et₂O to give a solid that was collected and dried. The solid was dissolved in H₂O and filtered through celite. H₂SO₄ (aq, 1 M, 1.84 mL, 1.84 mmol) was added to the filtrate, which was concentrated and dried in vacuo over P₂O₅. The residue was triturated with Et₂O to give the title compound (820 mg, 1.59 mmol, 86%).

[α]^{D}₂₀−65.7° (c=1.0, H₂O)

¹H NMR (400 MHz, D₂O): δ7.33 (d, 2H), 6.95 (d, 2H), 4.98 (t, 1H), 3.29 (d, 2H), 3.11 (t, 2H), 1.72-1.65 (m, 2H), 1.44-1.35 (m, 2H), 0.93 (t, 3H).

### Example 12

### 4-(2-(Butylamino)-1-hydroxypropyl)phenol acetate

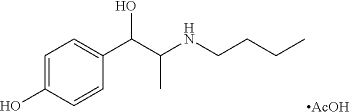 [SMILES: "·AcOH" ; CCCCNC(C)C(O)c1ccc(O)cc1]

(a) Phenyl propionate

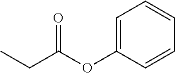

Solutions of tetra-n-butylammonium chloride (886 mg, 3.2 mmol) in CH₂Cl₂ (10 mL) and of propionyl chloride (2.8 mL, 32 mmol) in CH₂Cl₂ (30 mL), both at 0° C., were added to a solution of phenol (3.0 g, 32 mmol) in 10% NaOH (31 mL, 105 mmol) at 0° C. The mixture was stirred vigorously at 0° C. for 20 minutes and poured into ice/water. The layers were separated and the aq layer was extracted with Et₂O. The combined organic phases were washed with brine, dried over Na₂SO₄ and concentrated to give the sub-title compound (4.57 g, 30.4 mmol, 96%).

(b) 4-Hydroxypropiophenone

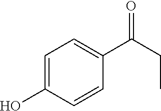

A mixture of phenyl propionate (1.0 g, 6.66 mmol) and trifluoromethanesulfonic acid (35 mL) was stirred at 0° C. for 30 minutes and at rt for 4 h and poured into cold water and ethyl acetate. CH₂Cl₂ was added and the layers were separated. The aq layer was extracted with CH₂Cl₂ and the combined organic phases were washed with 1M HCI (aq, 1M), sat NaHCO₃ (aq, sat), brine, dried over Na₂SO₄ and concentrated to give the sub-title compound (800 mg, 5.3 mmol, 80%).

(c) 4-Benzyloxypropiophenone

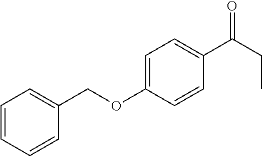 [SMILES: CCC(=O)c1ccc(OCc2ccccc2)cc1]

K₂CO₃ (1.47 g, 10.7 mmol) was added to a solution of 4-hydroxypropiophenone (0.80 g, 5.33 mmol) in acetone (40 mL) and the mixture was stirred for 30 minutes at rt. Benzylbromide (0.63 mL, 5.33 mmol) was added and the mixture was heated at reflux for 4 h and stirred at rt overnight. Filtration, concentration and crystallization from Et₂O/petroleum ether gave the sub-title compound (0.76 g, 3.16 mmol, 59%).

(d) 1-(4-(Benzyloxy)phenyl)-2-bromopropan-1-one

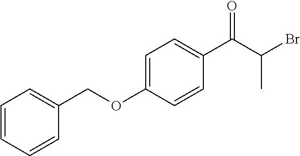 [SMILES: CC(Br)C(=O)c1ccc(OCc2ccccc2)cc1]

Br₂ (0.13 mL, 2.46 mmol) was added drop-wise to a solution of 4-benzyloxypropiophenone (590 mg, 2.46 mmol) in CH₂Cl₂. The mixture was stirred at rt for 40 min and concentrated. EtOAc was added to the residue, and the mixture was washed with 10% Na₂S₂O₃ (aq, 10%), NaHCO₃ (aq, sat), water, brine, and dried over Na₂SO₄. Concentration and crystallization from EtOAc/petroleum ether gave the sub-title compound (450 mg, 1.41 mmol, 57%).

(e) 1-(4-(Benzyloxy)phenyI)-2-(butylamino)propan-1-ol

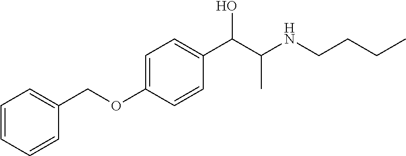 [SMILES: CCCCNC(C)C(O)c1ccc(OCc2ccccc2)cc1]

A mixture of 1-(4-(benzyloxy)phenyl)-2-bromopropan-1-one (220 mg, 0.69 mmol) and butylamine (0.68 mL, 6.89 mmol) was stirred at rt for 1.5 h. EtOH (4 mL) and NaBH₄ (78 mg, 2.07 mmol) was added and the mixture was stirred at rt for 1 h. NH₄Cl (aq, sat) was added and the mixture was stirred at rt for 10 minutes. The mixture was concentrated to remove the organic solvents and the aq residue was extracted with EtOAc. The combined organic extracts were washed with H₂O, brine, dried over Na₂SO₄, concentrated and purified by chromatography to give the sub-title compound (172 mg, 0.55 mmol, 80%).

(f) 4-(2-(Butylamino)-1-hydroxypropyl)phenol acetate

Hydrogenation of 1-(4-(benzyloxy)phenyl)-2-(butylamino)propan-1-ol (65 mg, 0.21 mmol) in accordance with the procedure in Example 7, Step (b) gave the title compound (52 mg, 0.18 mmol, 88%).

¹H NMR (300 MHz, D₂O) δ7.38-7.22 (m, 2H), 7.05-6.86 (m, 2H), 5.07 (d, J=3.1 Hz, 1H), 3.65-3.45 (m, 1H), 3.25-3.00 (m, 2H), 1.92 (s, 3H), 1.79-1.55 (m, 2H), 1.40 (sextet, J=7.3 Hz, 2H), 1.16 (d, J=6.7 Hz, 3H), 0.94 (t, J=7.3 Hz, 3H).

### Example 13

### 4-(1-Hydroxy-2-(pentan-2-ylamino)propyl)phenol acetate

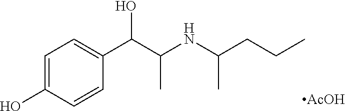 [SMILES: "·AcOH" ; CCCC(C)NC(C)C(O)c1ccc(O)cc1]

The title compound was prepared in accordance with Example 12, using 2-aminopentane in Step (e). It was obtained as 1:1 mixture of diastereomers.

¹H NMR (300 MHz, D₂O) δ7.39-7.25 (m, 4H), 7.03-6.89 (m, 4H), 5.06 (t, J=3.5 Hz, 2H), 3.76-3.61 (m, 2H), 3.54-3.35 (m, 2H), 1.92 (s, 6H), 1.83-1.67 (m, 2H), 1.66-1.37 (m, 6H), 1.36 (d, J=6.7 Hz, 3H), 1.33 (d, J=6.7 Hz, 3H), 1.16 (d, J=6.7 Hz, 6H), 0.95 (t, J=7.3 Hz, 3H), 0.93 (t, J=7.3 Hz, 3H).

### Example 14

### 3-(2-(Butylamino)-1-hydroxyethyl)phenol

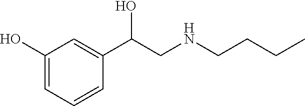

The title compound was obtained from 3-methoxyphenacyl bromide and butylamine in accordance with Example 12, Step (e) and Example 1, Step (c). It was obtained as ˜1:1 mixture of diastereomers.

¹H NMR (400 MHz, Acetone-d₆) δ8.44 (br s, 2H), 7.16 (t, J=7.8 Hz, 1H), 7.00-6.99 (m, 1H), 6.94-6.89 (m, 1H), 6.77 (ddd, J=8.1, 2.5, 1.0 Hz, 1H), 5.40 (br s, 1H), 5.32 (dd, J=10.1, 2.7 Hz, 1H), 3.35 (dd, J=12.6, 2.7 Hz, 1H), 3.24-3.12 (m, 3H), 1.98-1.85 (m, 2H), 1.52-1.39 (m, 2H), 0.94 (t, J=7.4 Hz, 3H).

### Example 15

### 3-(1-Hydroxy-2-(pentan-2-ylamino)ethyl)phenol

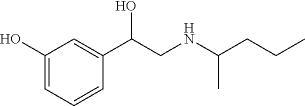 [SMILES: CCCC(C)NCC(O)c1cccc(O)c1]

The title compound was obtained from 3-methoxyphenacyl bromide and 2-aminopentane in accordance with Example 12, Step (e) and Example 1, Step (c). It was obtained as ˜1:1 mixture of diastereomers.

¹H NMR (400 MHz, Methanol-d₄) δ7.24-7.20 (m, 2H), 6.91-6.89 (m, 2H), 6.75 (dd, J=2.5, 1.0, 1H)/6.74 (dd, J=2.5, 1.0, 1H), 4.93 (dd, J=5.9, 3.2, 1H)/4.90 (dd, J=5.9, 3.2, 1H), 3.39-3.31 (m, 2H), 3.20 (dd, J=5.2, 3.2, 1H)/3.17 (dd, J=5.2, 3.2, 1H), 3.09 (dd, J=10.2, 1.5, 1H)/(3.06 (dd, J=10.2, 1.5), 1.85-1.75 (m, 2H), 1.64-1.46 (m, 4H), 1.46-1.39 (m, 2H), 1.34 (d, J=5.9 Hz, 3H)/1.32 (d, J=5.9 Hz, 3H), 1.00 (t, J=7.2, 3H)/0.99 (t, J=7.2, 3H).

### Example 16

### 4-(2-((2-Cyclohexylethyl)amino)-1-hydroxyethyl)phenol acetate

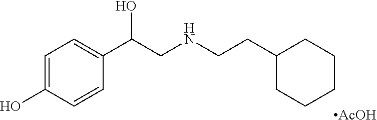 [SMILES: "·AcOH" ; Oc1ccc(C(O)CNCCC2CCCCC2)cc1]

The title compound was prepared from 4-benzyloxyphenacyl bromide (see Example 3, Step (b)) and cyclohexylethylamine in accordance with the procedures in Example 12, Step (e) and Example 7, Step (b).

¹H NMR (300 MHz, DMSO-d₆) δ7.18-7.03 (m, 2H), 6.78-6.61 (m, 2H), 4.53 (t, J=6.1 Hz, 1H), 2.65-2.53 (m, 4H), 1.84 (s, 3H), 1.71-1.52 (m, 5H), 1.37-1.01 (m, 6H), 0.96-0.75 (m, 2H).

### Example 17

### 4-(2-(Hexylamino)-1-hydroxyethyl)phenol acetate

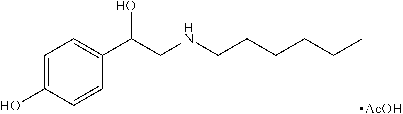 [SMILES: "·AcOH" ; CCCCCCNCC(O)c1ccc(O)cc1]

The title compound was prepared from 4-benzyloxyphenacyl bromide (see Example 3, Step (b)) and hexylamine in accordance with the procedures in Example 12, Step (e) and Example 7, Step (b).

¹H NMR (400 MHz, THF-d₈) δ7.22-7.04 (m, 2H), 6.76-6.56 (m, 2H), 5.84 (br s, 4H), 4.66 (br s, 1H), 2.86-2.51 (m, 4H), 1.88 (br s, 3H), 1.53 (br s, 2H), 1.33 (br s, 6H), 0.89 (br s, 3H).

### Example 18

### 4-(1-Hydroxy-2-(octylamino)ethyl)phenol acetate

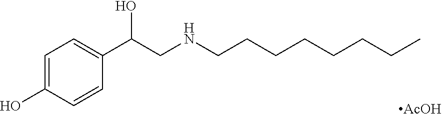 [SMILES: "·AcOH" ; CCCCCCCCNCC(O)c1ccc(O)cc1]

The title compound was prepared from 4-benzyloxyphenacyl bromide (see Example 3, Step (b)) and octylamine in accordance with the procedures in Example 12, Step (e) and Example 7, Step (b).

¹H NMR (400 MHz, THF-d₈) δ7.13 (br s, 2H), 6.66 (br s, 2H), 5.03-4.25 (m, 5H), 2.80-2.37 (m, 4H), 1.89 (br s, 3H), 1.48 (br s, 2H), 1.30 (br s, 10H), 0.89 (br s, 3H).

### Example 19

### 2-Chloro-4-(1-hydroxy-2-(pentan-2-ylamino)ethyl)phenol

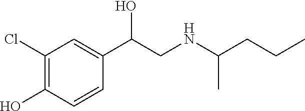 [SMILES: CCCC(C)NCC(O)c1ccc(O)c(Cl)c1]

The title compound was prepared from 3-chloro-4-methoxyacetophenone and 2-amino-pentane in accordance with the procedures in Example 12, Steps (d) and (e) and Example 1, Step (c). It was obtained as -1:1 mixture of diastereomers.

¹H NMR (400 MHz, DMSO-d₆) δ7.26 (d, J=2.1 Hz, 1H), 7.07 (dd, J=8.4, 2.1 Hz, 1H), 6.89 (d, J=8.3 Hz, 1H), 5.27 (br s, 2H), 4.50-4.44 (m, 1H), 2.68-2.52 (m, 3H), 1.40-1.13 (m, 4H), 0.94 (dd, J=7.4, 6.2 Hz, 3H), 0.85 (td, J=7.1, 5.2 Hz, 3H).

### Example 20

### 4-(2-(Butylamino)-1-hydroxyethyl)-2-chlorophenol

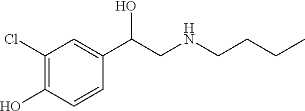 [SMILES: CCCCNCC(O)c1ccc(O)c(Cl)c1]

The title compound was prepared from 3-chloro-4-methoxyacetophenone and butylamine in accordance with the procedures in Example 12, Steps (d) and (e) and Example 1, Step (c).

¹H NMR (400 MHz, DMSO-d₆) δ7.25 (d, J=2.1 Hz, 1H), 7.06 (dd, J=8.4, 2.1 Hz, 1H), 6.89 (d, J=8.3 Hz, 1H), 5.41 (br s, 3H), 4.51 (t, J=6.3 Hz, 1H), 2.58 (d, J=6.3 Hz, 2H), 2.56-2.51 (m, 2H), 1.42-1.22 (m, 4H), 0.86 (t, J=7.2 Hz, 3H).

### Example 21

### 4-(2-((3-Cyclohexylpropyl)amino)-1-hydroxyethyl)phenol

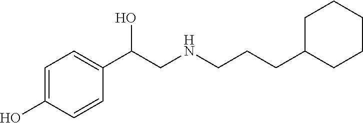 [SMILES: Oc1ccc(C(O)CNCCCC2CCCCC2)cc1]

(a) 1-(4-(Benzyloxy)phenyI)-2-((3-cyclohexylpropyl)amino)ethan-1-ol

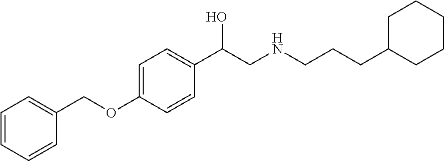 [SMILES: OC(CNCCCC1CCCCC1)c1ccc(OCc2ccccc2)cc1]

DIPEA (0.24 mL, 1.36 mmol) was added dropwise to a mixture of (3-bromopropyl)-cyclohexane hydrobromide (303 mg, 1.36 mmol) and CH₂Cl₂ (4 mL) at 0° C. A solution of 4-benzyloxyphenacyl bromide (208 mg, 0.68 mmol, see Example 3, Step (b)) in CH₂Cl₁₂ (3 mL) was added over 15 min to the stirred mixture at 0° C. The mixture was stirred at 0° C. for 1 h and at 5° C. overnight. EtOH (5 mL) followed by NaBH₄ (77 mg, 2.04 mmol) was added at 0° C. and the mixture was stirred at 0° C. for 30 min and at rt for 30 min. NH₄Cl (aq, sat, 5 mL) was added and after gas evolution had ceased, the mixture was concentrated to remove the organic solvents and the aq residue was extracted with EtOAc. The combined organic extracts were washed with H2O, brine, dried over Na₂SO₄, concentrated and purified by chromatography to give the sub-title compound (80mg, 0.22 mmol, 32%).

(b) 4-(2-((3-Cyclohexylpropyl)amino)-1-hydroxyethyl)phenol

1-(4-(Benzyloxy)phenyl)-2-((3-cyclohexylpropyl)amino)ethan-1-ol (50 mg, 0.14 mmol) was hydrogenated in accordance with the procedure in Example 7, Step (b), followed by purification by chromatography to give the title compound (10 mg, 0.036 mmol, 27%).

¹H NMR (300 MHz, D₂O+a drop of TFA) 8 7.28-7.14 (m, 2H), 6.88-6.76 (m, 2H), 4.86 (t, J=6.7 Hz, 1H), 3.17 (d, J=6.6 Hz, 2H), 2.96 (t, J=7.9 Hz, 2H), 1.68-1.40 (m, 7H), 1.22-0.93 (m, 6H), 0.87-0.65 (m, 2H).

### Example 22

### 4-(2-((2-Cyclopropylethyl)amino)-1-hydroxyethyl)phenol

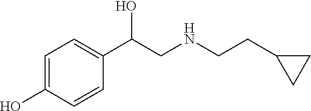 [SMILES: Oc1ccc(C(O)CNCCC2CC2)cc1]

The title compound was obtained from 4-methoxyphenacyl bromide and cyclopropyl-ethylamine in accordance with Example 12, Step (e) and Example 1, Step (c).

¹H NMR (400 MHz, DMSO-d₆) δ9.20 (br s, 1H), 7.14-7.08 (m, 2H), 6.72-6.65 (m, 2H), 5.03 (br s, 1H), 4.49 (dd, J=7.7, 5.0 Hz, 1H), 2.65-2.52 (m, 4H), 1.28 (q, J=7.1 Hz, 2H), 0.72-0.60 (m, 1H), 0.40-0.31 (m, 2H), 0.03-0.04 (m, 2H).

### Example 23

### 4-(2-((3-Cyclopropylpropyl)amino)-1-hydroxyethyl)phenol acetate

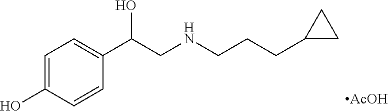 [SMILES: "·AcOH" ; Oc1ccc(C(O)CNCCCC2CC2)cc1]

(a) 1-(4-(Benzyloxy)phenyI)-2-((3-cyclopropylpropyl)amino)ethan-1-ol

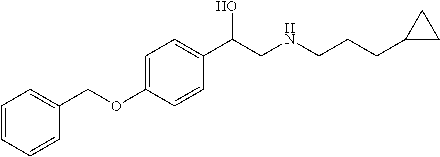 [SMILES: OC(CNCCCC1CC1)c1ccc(OCc2ccccc2)cc1]

A solution of 4-benzyloxyphenacyl bromide (195 mg, 0.64 mmol, see Example 3, Step (b)) in CH₂Cl₂ (5 mL) was added dropwise to a mixture 3-cyclopropylpropylamine (127 mg, 1.28 mmol), DIPEA (0.11 mL, 0.64 mmol) and CH₂Cl₂ (5 mL) at −20° C. The mixture was stirred at −20° C. for 1 h and kept at 31 20° C. overnight. MeOH (5 mL) followed by NaBH₄ (97 mg, 2.56 mmol) was added and the mixture was stirred at rt for 1 h and concentrated.

Water was added to the residue and the mixture was extracted with EtOAc. The combined organic extracts were washed with H₂O, brine, dried over Na₂SO₄, concentrated and purified by chromatography to give the sub-title compound (90mg, 0.28 mmol, 43%).

(b) 4-(2-((3-Cyclopropylpropyl)amino)-1-hydroxyethyl)phenol acetate

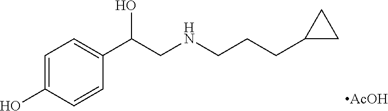 [SMILES: "·AcOH" ; Oc1ccc(C(O)CNCCCC2CC2)cc1]

Hydrogenation of 1-(4-(benzyloxy)phenyI)-2-((3-cyclopropylpropyl)amino)ethan-1-ol (81 mg, 0.25 mmol) in accordance with the procedure in Example 7, Step (b) gave the title compound (42 mg, 0.14 mmol, 58%).

¹H NMR (400 MHz, D₂O) δ7.37-7.30 (m, 2H), 6.98-6.92 (m, 2H), 5.02-4.94 (m, 1H), 3.30 (d, J=6.5 Hz, 2H), 3.19-3.13 (m, 2H), 1.92 (s, 3H), 1.87-1.77 (m, 2H), 1.29 (q, J=7.2 Hz, 1H), 0.78-0.64 (m, 1H), 0.48-0.40 (m, 2H), 0.10-0.01 (m, 2H).

### Example 24

### 4-(1-Hydroxy-2-((4,4,4-trifluorobutyl)amino)ethyl)phenol acetate

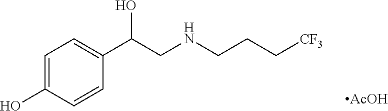 [SMILES: "·AcOH" ; Oc1ccc(C(O)CNCCCC(F)(F)F)cc1]

The title compound was prepared from 4-benzyloxyphenacyl bromide and 4,4,4-trifluoro-butan-1-amine in accordance with the procedure in Example 23.

¹H NMR (400 MHz, D₂O) δ7.38c-7.28 (m, 2H), 6.98-6.89 (m, 2H), 5.03-4.93 (m, 1H), 3.32 (d, J=6.6 Hz, 2H), 3.25-3.17 (m, 2H), 2.41-2.26 (m, 2H), 2.08-1.95 (m, 2H), 1.91 (s, 3H).

### Example 25

### 5-(2-(Butylamino)-1-hydroxyethyl)benzene-1,3-diol hemisulphate

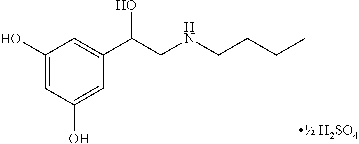 [SMILES: CCCCNCC(O)c1cc(O)cc(O)c1]

(a) 1-(3,5-bis(benzyloxy)phenyl)-2-(butylamino)ethan-1-ol

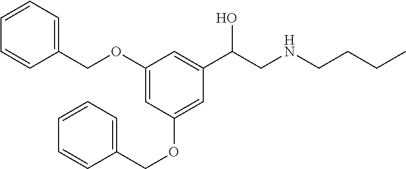 [SMILES: CCCCNCC(O)c1cc(OCc2ccccc2)cc(OCc2ccccc2)c1]

The sub-title compound was prepared from 3,5-dibenzyloxyacetophenone in accordance with the procedures in Example 12, Steps (d) and (e).

(b) 5-(2-(Butylamino)-1-hydroxyethyl)benzene-1,3-diol hemisulphate

A mixture of 1-(3,5-bis(benzyloxy)phenyl)-2-(butylamino)ethan-1-ol (170 mg, 0.42 mmol), 10% Pd—C (89 mg, 0.084 mmol) and AcOH (5 mL) was hydrogenated at 9 atm at rt for 2 h, filtered through Celite and concentrated. The residue was purified by chromatography to give an oil (45 mg) that was dissolved in H₂O and 0.5 M H₂SO₄ (1M, 0.08 mL). The solution was concentrated, treated with Et₂O and concentrated. The residue was again treated with Et₂O and concentrated to give the title compound (45 mg, 0.082 mmol, 20%).

¹H NMR (400 MHz, D₂O) δ6.51-6.49 (m, 2H), 6.40-6.39 (m, 1H), 4.93 (dd, J=9.2, 3.7 Hz, 1H), 3.33-3.18 (m, 2H), 3.14-3.05 (m, 2H), 1.72-1.64 (m, 2H), 1.44-1.34 (m, 2H), 0.93 (t, J=7.4 Hz, 3H).

### Example 26

### 4-(2-(Butylamino)-1-hydroxyethyl)-2,6-dichlorophenol

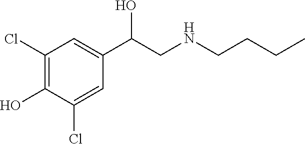 [SMILES: CCCCNCC(O)c1cc(Cl)c(O)c(Cl)c1]

(a) 2,6-Dichlorophenyl acetate

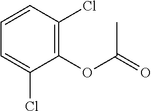

Acetyl chloride (2.1 mL, 2.3 g, 29.4 mmol) was added dropwise to a mixture of 2,6-dichlorophenol (3.7 mL, 4 g, 24.5 mmol), Et₃N (8.6 mL, 6.2 g, 61.3 mmol) and CH₂Cl₂ (30 mL) at 0° C. The mixture was stirred at rt for 2.5 h, treated with Na₂CO₃ (aq, sat) and extracted with CH₂Cl₂. The combined extracts were washed with brine, dried over Na₂SO₄ and concentrated to give the sub-title compound (4.75 g, 23.1 mmol, 94%).

(b) 3,5-Dichloro-4-hydroxyacetophenone

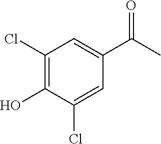

A mixture of 2,6-dichlorophenyl acetate (4.75 g, 23.2 mmol) and trifluoromethanesulfonic acid (10 mL) was stirred at 40° C. for 16 h and cooled in an ice-bath. The mixture was neutralized by carefull addition of Na₂CO₃ (aq, sat) and extracted with EtOAc. The combined extracts were washed with brine, dried over Na₂SO₄ and concentrated to give the sub-title compound (2.2 g, 10.7 mmol, 46%).

(c) 3,5-Dichloro-4-methoxyacetophenone

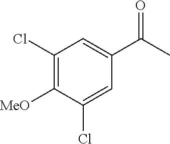

K₂CO₃ (1.21 g, 8.8 mmol) was added to a mixture of 3,5-dichloro-4-hydroxyacetophenone (1.5 g, 7.3 mmol) and DMF (10 mL). Methyl iodide (0.46 mL, 7.3 mmol) was slowly added at rt and the mixture was stirred at rt for 3 h. The mixture was poured into water and extracted with Et₂O. The combined extracts were washed with brine, dried over Na₂SO₄ and concentrated to give the sub-title compound (300 mg, 1.4 mmol, 19%).

(d) 4-(2-(Butylamino)-1-hydroxyethyl)-2,6-dichlorophenol

The title compound was prepared from 3,5-dichloro-4-methoxyacetophenone and n-butylamine in accordance with the procedures in Example 12, Steps (d) and (e), and Example 1, Step (c).

¹H NMR (400 MHz, DMSO-d₆) δ7.35 (s, 2H), 4.80 (dd, J=9.6, 3.2 Hz, 1H), 3.09 (dd, J=12.6, 3.3 Hz, 1H), 2.95 (dd, J=12.6, 9.7 Hz, 1H), 2.90-2.83 (m, 2H), 1.62-1.52 (m, 2H), 1.35-1.26 (m, 2H), 0.88 (t, J=7.4 Hz, 3H).

### Example 27

### 2,6-Dichloro-4-(1-hydroxy-2-(pentan-2-ylamino)ethyl)phenol

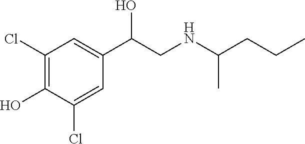 [SMILES: CCCC(C)NCC(O)c1cc(Cl)c(O)c(Cl)c1]

The title compound was prepared in accordance with the procedures in Example 28, using 2-aminopentane, and was obtained as an -1:1 mixture of diastereomers.

¹H NMR (400 MHz, D₂O+1 drop of TFA) δ7.27 (s, 4H), 4.82-4.81 (m, 2H, overlapping with D₂O), 3.28-3.21 (m, 2H), 3.20-3.02 (m, 4H), 1.64-1.53 (m, 2H), 1.49-1.37 (m, 2H), 1.34-1.20 (m, 4H), 1.18 (d, J=6.6 Hz, 3H), 1.18 (d, J=6.6 Hz, 3H), 0.78 (t, J=7.3 Hz, 6H).

### Example 28

### 1-(4-Amino-3,5-dichlorophenyl)-2-(pentan-2-ylamino)ethan-1-ol trifluoroacetate

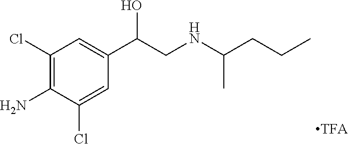 [SMILES: CCCC(C)NCC(O)c1cc(Cl)c(N)c(Cl)c1]

(a) N,N-Bis(tert-butoxycarbonyl)-2,6-dichloro-4-acetylaniline

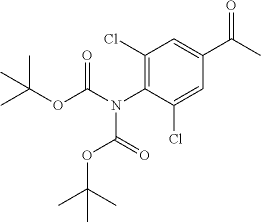 [SMILES: CC(=O)c1cc(Cl)c(N(C(=O)OC(C)(C)C)C(=O)OC(C)(C)C)c(Cl)c1]

Boc₂O (1.76 g, 7.4 mmol) and DMAP (90 mg, 0.7 mmol) was added to a solution of 4-amino-3,5-dichloroacetophenone (1.5 g, 7.4 mmol) in THF (15 mL) at rt. The mixture was heated at reflux for 2 h and cooled to rt. Another portion of Boc₂O (1.76 g, 7.4 mmol) and DMAP (90 mg, 0.7 mmol) was added and the mixture was stirred at rt for 2 d. The mixture was, concentrated, diluted with EtOAc and washed with citric acid (aq, 2 M). The layers were separated and the aq phase was extracted with EtOAc. The combined organic phases were washed with brine, dried over Na₂SO₄, concentrated and purified by chromatography to give the sub-title compound (1.67 g, 4.1 mmol, 56%).

(b) 1-(N,N-Bis(tert-butoxycarbonyl)-4-amino-2,6-dichlorophenyl)vinyl tent-butyl carbonate

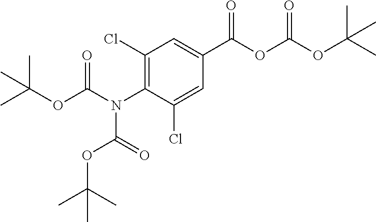 [SMILES: CC(C)(C)OC(=O)OC(=O)c1cc(Cl)c(N(C(=O)OC(C)(C)C)C(=O)OC(C)(C)C)c(Cl)c1]

Boc₂O (1.35 g, 6.2 mmol) and DMAP (51 mg, 0.4 mmol) was added to a solution of N,N-bis(tert-butoxycarbonyl)-2,6-dichloro-4-acetylaniline (1.67 g, 4.1 mmol) in THF (20 mL) at rt. The mixture was stirred at rt and four additional portions of Boc₂O (1.35 g, 6.2 mmol) and DMAP (51 mg, 0.4 mmol) were added over 5 days. The mixture was diluted with EtOAc and washed with citric acid (aq, 2 M). The phases were separated and the aq layer was extracted with EtOAc. The combined organic layers were washed with brine, dried over Na2SO₄, concentrated and purified by chromatography to give the sub-title compound (1.38 g, 2.7 mmol, 66%).

(c) N,N-Bis(tert-butoxycarbonyl)-2,6-dichloro-4-(bromoacetyl)aniline

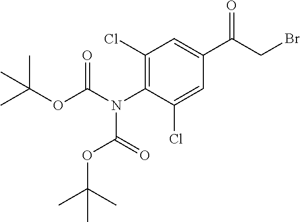 [SMILES: CC(C)(C)OC(=O)N(C(=O)OC(C)(C)C)c1c(Cl)cc(C(=O)CBr)cc1Cl]

N-Bromosuccinimide (530 mg, 3.0 mmol) and H₂O (54 μL, 3.0 mmol) was added to a mixture of 1-(N,N-bis(tert-butoxycarbonyl)-4-amino-2,6-dichlorophenyl)vinyl tent-butyl carbonate (1 g, 2.0 mmol) and THF (20 mL) at rt. The mixture was stirred at rt for 3 d. H₂O was added and the mixture was extracted with EtOAc. The combined extracts were washed with brine, dried over Na₂SO₄, concentrated and purified by chromatography to give the sub-title compound (670 mg, 1.4 mmol, 70%).

(d) N,N-bis(tert-butoxycarbonyl)-2,6-dichloro-4-(1-hydroxy-2-(2-pentylamino)-ethyl)aniline

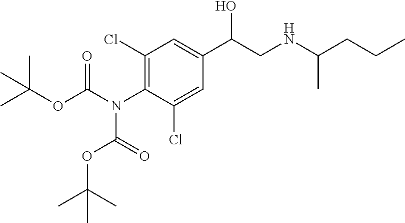 [SMILES: CCCC(C)NCC(O)c1cc(Cl)c(N(C(=O)OC(C)(C)C)C(=O)OC(C)(C)C)c(Cl)c1]

The sub-title compound was prepared from N,N-bis(tert-butoxycarbonyl)-2,6-dichloro-4-(bromoacetyl)aniline and 2-aminopentane in accordance with the procedure in Example 12, Step (e).

(e) 1-(4-Amino-3,5-dichlorophenyl)-2-(pentan-2-ylamino)ethan-1-ol trifluoroacetate

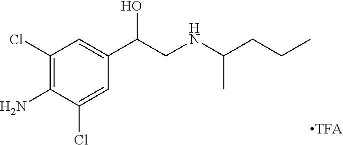 [SMILES: CCCC(C)NCC(O)c1cc(Cl)c(N)c(Cl)c1]

TFA (0.46 mL, 5.9 mmol) was added to a solution of N,N-bis(tert-butoxycarbonyl)-2,6-dichloro-4-(1-hydroxy-2-(2-pentylamino)ethyl)aniline in CH₂Cl₂ (4 mL) at rt. The mixture was stirred at rt for 1 h and an additional portion of TFA (0.23 mL, 3.0 mmol) was added. After stirring at rt for 1 h, the mixture was concentrated and purified by chromatography to give the title compound (10 mg, 0.025 mmol, 17%). The compound is a 1:1 mixture of diastereomers.

¹H NMR (400 MHz, DMSO-d₆) δ8.66-8.13 (m, 4H), 7.28 (s, 4H), 6.22-6.05 (m, 2H), 5.54 (s, 4H), 4.81-4.66 (m, 2H), 3.24-2.92 (m, 6H), 1.78-1.59 (m, 2H), 1.51-1.31 (m, 4H), 1.30-1.23 (m, 2H), 1.20 (d, J=5.0 Hz, 3H), 1.19 (d, J=5.0 Hz) 0.89 (t, J=7.3 Hz, 3H), 0.89 (t, J=7.3 Hz).

### Example 29

### 1-(4-Amino-3,5-dichlorophenyl)-2-(butylamino)ethan-1-ol trifluoroacetate

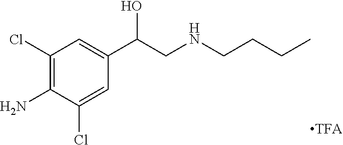 [SMILES: CCCCNCC(O)c1cc(Cl)c(N)c(Cl)c1]

(a) 4-Amino-3,5-dichlorophenacyl bromide

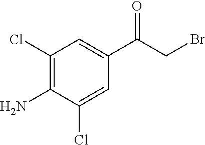 [SMILES: Nc1c(Cl)cc(C(=O)CBr)cc1Cl]

Br₂ (0.33 mL, 1.0 g, 6.4 mmol) in CHCl₃ (10 mL) was added over 10 min to a mixture of 4-amino-3,5-dichloroacetophenone (1 g, 4.9 mmol) in CHCl₃ (20 mL) at reflux. The mixture was heated at reflux for 15 min and allowed to cool to rt. The mixture was concentrated and THF (20 mL) was added. A solution of diethyl phosphite (537 mg, 3.9 mmol) and Et₃N (0.55 mL, 3.9 mmol) in THF (5 mL) was slowly added at 0° C. and the temperature was allowed to come to rt. The mixture was stirred for 10 min, concentrated and poured onto ice. The solid was filtered off, washed with water and dried to give the sub-title compound (1.24 g, 4.4 mmol, 89%).

(b) 1-(4-Amino-3-chloro-5-(trifluoromethyl)phenyl)-2-(butylamino)ethan-1-ol

The title compound was prepared from 1-(4-amino-3,5-dichlorophenyl)-2-bromoethan-1-one and n-butylamine in accordance with the procedure in Example 12, Step (e).

¹H NMR (400 MHz, cdcl₃) δ7.20 (s, 1H), 4.52 (dd, J=9.1, 3.7 Hz, 1H), 4.40 (s, 2H), 2.84 (dd, J=12.2, 3.7 Hz, 1H), 2.72-2.55 (m, 3H), 1.52-1.42 (m, 2H), 1.40-1.29 (m, 2H), 0.92 (t, J=7.3 Hz, 3H).

### Example 30

### 1-(4-Amino-3-chloro-5-(trifluoromethyl)phenyl)-2-(butylamino)ethan-1-ol

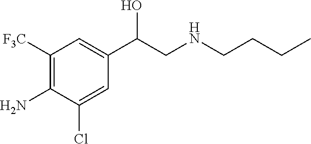 [SMILES: CCCCNCC(O)c1cc(Cl)c(N)c(C(F)(F)F)c1]

The title compound was prepared from 4-acetyl-2-chloro-6-trifluoromethylaniline and n-butylamine in accordance with the procedures in Example 29, Step (a), and Example 12, Step (e).

¹H NMR (400 MHz, CDCl₃) δ7.46 (d, J=1.8 Hz, 1H), 7.36 (d, J=1.8 Hz, 1H), 4.63-4.54 (m, 3H), 2.87 (dd, J=12.2, 3.6 Hz, 1H), 2.74-2.57 (m, 3H), 1.53-1.44 (m, 2H), 1.42-1.30 (m, 2H), 0.92 (t, J=7.3 Hz, 3H).

### Example 31

### 1-(4-amino-3-chloro-5-(trifluoromethyl)phenyl)-2-(pentan-2-ylamino)ethan-1-ol

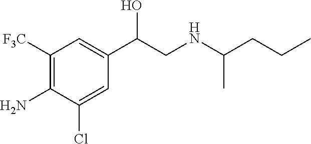 [SMILES: CCCC(C)NCC(O)c1cc(Cl)c(N)c(C(F)(F)F)c1]

The title compound was prepared from 4-acetyl-2-chloro-6-trifluoromethylaniline and 2-aminopentane in accordance with the procedures in Example 29, Step (a), and Example 12, Step (e). The compound is a 1:1 mixture of diastereomers.

¹H NMR (400 MHz, CDCl₃) δ7.47 (s, 1H), 7.36 (s, 1H), 4.60 (br s, 2H), 4.54 (dd, J=9.3, 3.5 Hz, 1H), 2.96 (dd, J=12.2, 3.6 Hz, 0.5H), 2.90 (dd, J=12.2, 3.6 Hz, 0.5H), 2.73-2.63 (m, 1H), 2.60 (dd, J=12.3, 9.2 Hz, 0.5H), 2.52 (dd, J=12.2, 9.3 Hz, 0.5H), 1.50-1.23 (m, 4H), 1.08 (d, J=1.9 Hz, 1.5H), 1.07 (d, J=1.9 Hz, 1.5H), 0.91 (t, J=7.0 Hz, 3H).

### Example 32

### 5-(2-(Butylamino)-1-hydroxyethyl)-2-chlorophenol

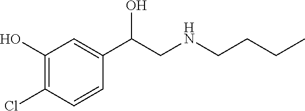 [SMILES: CCCCNCC(O)c1ccc(Cl)c(O)c1]

(a) 1-(4-Chloro-3-methoxyphenyl)ethan-1-ol

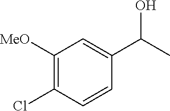

MeMgBr (1 M in THF, 9.67 mL, 9.67 mmol) was added to a solution of of 4-chloro-3-methoxybenzaldehyde (1.50 g, 8.79 mmol) in THF (10 mL) at −78° C. The mixture was stirred at −78° C. for 10 min and at rt for 3 h. NH₄Cl (aq, sat, 20 mL) was carefully added and the mixture was extracted with EtOAc. The combined extracts were washed with H₂O and brine, and dried over Na₂SO₄. Concentration and purification by chromatography gave the sub-title compound (1.10 g, 5.89 mmol, 67%).

(b) 4-Chloro-3-methoxyacetophenone

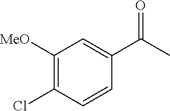

Dess-Martin periodinane (3.00 g, 7.07 mmol) in CH₂Cl₂ (25 mL) was slowly to a solution of 1-(4-chloro-3-methoxyphenyl)ethan-1-ol in CH₂Cl₂ (25 mL) at rt. The mixture was stirred at rt for 1 h. NaOH (1 M, aq, 30 mL) was added and the mixture was stirred for 30 min. The layers were separated and the organic phase was washed with NaOH (1 M, aq), H₂O and brine, and dried over Na₂SO₄. Concentration and purification by chromatography gave the sub-title compound (0.91 g, 4.92 mmol, 84%).

(c) 5-(2-(Butylamino)-1-hydroxyethyl)-2-chlorophenol

The title compound was prepared from 4-chloro-3-methoxyacetophenone and n-butylamine in accordance with the procedures in Example 29, Step (a), Example 12, Step (e), and Example 1, Step (c).

¹H NMR (400 MHz, D₂O) δ7.45 (d, J=8.2 Hz, 1H), 7.07-7.06 (m, 1H), 6.97 (ddd, J=8.2, 2.0, 0.6 Hz, 1H), 5.00 (dd, J=9.2, 3.8 Hz, 1H), 3.33 (dd, J=13.1, 3.7 Hz, 1H), 3.26 (dd, J=13.0, 9.4 Hz, 1H), 3.12-3.07 (m, 2H), 1.73-1.66 (m, 2H), 1.45-1.35 (m, 2H), 0.94 (t, J=7.4 Hz, 3H).

### Example 33

### 2-Chloro-5-(1-hydroxy-2-(pentan-2-ylamino)ethyl)phenol

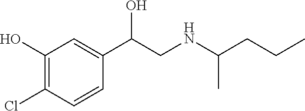 [SMILES: CCCC(C)NCC(O)c1ccc(Cl)c(O)c1]

The title compound was prepared from 4-chloro-3-methoxybenzaldehyde and 2-aminopentane in accordance with the procedures in Example 32. The compound is a 1:1 mixture of diastereomers.

¹H NMR (400 MHz, D₂O) δ7.46 (d, J=8.3 Hz, 2H, 7.08-7.07 (m, 2H), 7.00-6.95 (m, 2H), 5.01-4.96 (m, 2H), 3.44-3.37 (m, 2H), 3.36-3.30 (m, 2H), 3.28-3.21 (m, 2H), 1.79-1.69 (m, 2H), 1.64-1.52 (m, 2H), 1.50-1.41 (m, 2H), 1.40-1.35 (m, 2H), 1.33 (d, J=6.6 Hz, 3H), 1.33 (d, J=6.6 Hz, 3H), 0.94 (t, J=7.3 Hz, 3H), 0.94 (t, J=7.3 Hz, 3H).

### Example 34

### 1-(3-Amino-4-chlorophenyl)-2-(butylamino)ethan-1-ol

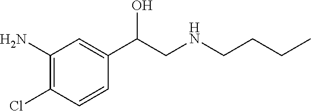 [SMILES: CCCCNCC(O)c1ccc(Cl)c(N)c1]

(a) 2-Bromo-1-(4-chloro-3-nitrophenyl)ethan-1-ol

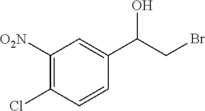 [SMILES: O=[N+]([O-])c1cc(C(O)CBr)ccc1Cl]

NaBH₄ (23.8 mg, 0.63 mmol) was added in portions to a mixture of 4-chloro-3-nitrophenacyl bromide (500 mg, 1.80 mmol) and MeOH (5 mL) at 0° C. The mixture was stirred at rt for 3 h and concentrated. H₂O was added to the residue and the mixture was extracted with Et₂O. The combined extracts were washed with NH₄Cl (aq, sat) and brine, dried over MgSO₄ and concentrated. The residue was purified by chromatography to give the sub-title compound (480 mg, 1.71 mmol, 95%).

(b) 2-(4-Chloro-3-nitrophenyl)oxirane

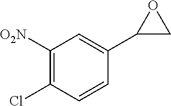 [SMILES: O=[N+]([O-])c1cc(C2CO2)ccc1Cl]

A mixture of 2-bromo-1-(4-chloro-3-nitrophenyl)ethan-1-ol (470 mg, 1.68 mmol), K₂CO₃ (347 mg, 2.51 mmol) and MeOH (17 mL) was stirred at rt for 30 min, diluted with H₂O and extracted with CH₂Cl₂. The combined extracts were washed with H₂O, dried over MgSO₄, filtered through neutral Al₂O₃ and concentrated to give the sub-title compound (286 mg, 1.43 mmol, 86%).

(c) 2-(Butylamino)-1-(4-chloro-3-nitrophenyl)ethan-1-ol

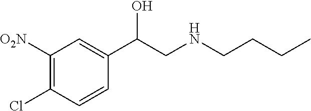 [SMILES: CCCCNCC(O)c1ccc(Cl)c([N+](=O)[O-])c1]

A mixture of 2-(4-chloro-3-nitrophenyl)oxirane (280 mg, 1.40 mmol), n-butylamine (139 μL, 1.40 mmol) and EtOH (4.5 mL) was stirred at 60° C. for 10 h and concentrated. The residue was purified by chromatography to give the sub-title compound (280 mg, 1.03 mmol, 73%).

(d) 1-(3-Amino-4-chlorophenyl)-2-(butylamino)ethan-1-ol

A mixture of iron powder (93.4 mg, 1.67 mmol), AcOH (54 μL) and H₂O (130 μL) was heated at 80° C. for 30 min. A mixture of 2-(butylamino)-1-(4-chloro-3-nitrophenyl)ethan-1-ol (120 mg, 0.44 mmol) in toluene (2.3 mL) and AcOH (aq, 20%, 1.6 mL) was added and the mixture was stirred at 80° C. for 2 h. The mixture was allowed to cool and filtered through Celite, which was washed with AcOH. Toluene was added to the filtrates and the layers were separated. The organic phase was washed with H₂O and the H₂O/AcOH phases were concentrated. NaHCO₃ (aq, sat) was added to the residue and the mixture was extracted with EtOAc. The combined extracts were dried over Na₂SO₄ and concentrated to give the titel compound (51 mg, 0.21 mmol, 48%). An analytical sample was obtained by crystallization from Et₂O and then from MeCN.

¹H NMR (400 MHz, CDCl₃) δ7.20-7.18 (d, J=8.2 Hz, 1H), 6.82 (d, J=1.6 Hz, 1H), 6.67-6.65 (d, J=9.9 Hz, 1H), 4.59-4.56 (dd, J=8.6, 3.4 Hz, 1H), 4.04 (s, 2H), 2.88-2.84 (dd, J=12.1, 3.5 Hz, 1H), 2.68-2.59 (m, 3H), 1.48-1.32 (dq, J=43.0, 7.4 Hz, 4H), 0.94-0.90 (t, J=7.3 Hz, 3H).

### Example 35

### 1-(4-Amino-3,5-difluorophenyl)-2-(butylamino)ethan-1-ol

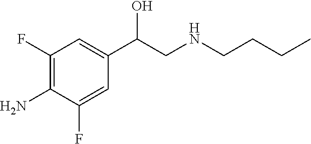 [SMILES: CCCCNCC(O)c1cc(F)c(N)c(F)c1]

(a) 4-Amino-3,5-difluoroacetophenone

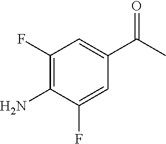

PdCl₂(MeCN)₂ (101.7 mg, 0.39 mmol) was added to a mixture of 2,6-difluoro-4-iodoaniline (2.0 g, 7.84 mmol), ZnO (830 mg, 10.2 mmol), Bu₄NBr (3.79 g, 11.8 mmol), Et₃N (372 82 L, 2.67 mmol) and DMSO (20 mL). The mixture was stirred at 100° C. for 16 h (not protected from air). Et₃N (47.4 μL, 0.34 mmol) was added and the mixture stirred for 6 h, cooled to rt, diluted with Et₂O and washed with H₂O. The aq layer was extracted with Et₂O and the combined extracts were washed with brine, dried over Na₂SO₄ and concentrated. Purification by chromatography gave the sub-title compound (270 mg, 1.58 mmol, 20%).

(b) 1-(4-Amino-3,5-difluorophenyl)-2-(butylamino)ethan-1-ol

The title compound was prepared from 4-amino-3,5-difluoroacetophenone and n-butylamine in accordance with the procedures in Example 29, Step (a) and Example 12, Step (e).

¹H NMR (400 MHz, CDCl₃) δ6.89-6.80 (m, 2H), 4.54 (dd, J=8.8, 3.4 Hz, 1H), 3.75-3.60 (br s, 2H), 2.84 (dd, J=12.1, 3.4 Hz, 1H), 2.70-2.57 (m, 3H), 2.73-2.03 (br s, 2H, overlapping), 1.51-1.41 (m, 2H), 1.40-1.29(m, 2H), 0.92 (t, J=7.4 Hz, 3H).

### Biological Examples

L6-myoblasts were grown in Dulbecco's Modified Eagle's Medium (DMEM) containing 4,5 g/I glucose supplemented with 10% fetal bovine serum, 2 mM L-Glutamine, 50 U/ml penicillin, 50 μg/ml streptomycin and 10 mM HEPES. Cells were plated at 1×10⁵ cells per ml in 24-well plates. After reaching 90% confluence the cells were grown in medium containing 2% FBS for 7 days where upon cells differentited into myotubes.

### Biological example 1

### Glucose Uptake

Differentiated L6-myotubes were serum-starved over night in medium containing 0.5% fatty-acid free BSA and stimulated with agonist, final concentration 1×10⁻⁵. After 1 h 40 min cells were washed with warm, glucose free medium or PBS and another portion of agonist was added to glucose free medium. After 20 min the cells were exposed to 50 nM ³H-2-deoxy-glucose for another 10 min before washed in ice cold glucose free medium or PBS and lysed in 0.2 M NaOH for 1 h in 60° C. Cell lysate was mixed with scintillation buffer (Emulsifier Safe, Perkin Elmer and radioactivity detected in a β-counter (Tri-Carb 2800TR, Perkin Elmer). The numerical values in the table are given as % increase over the basal level.

### Biological Example 2

### Measurement of Intracellular cAMP Levels

Differentiated cells were serum-starved over night and stimulated with agonist, final concentration 1×10−⁵, for 15 min in stimulation buffer (HBSS supplemented with 1% BSA, 5 mM HEPES and 1 mM IBMX, pH 7,4) The medium was then aspirated and to end the reaction 100 μL of 95% EtOH was added to each well of a 24-well plate and cells were kept in −20° C. over night. The EtOH was let to evaporate and 500 μL of lysis buffer (1% BSA, 5 mM HEPES and 0,3% Tween-20, pH 7.4) was added to each well before put in −80° C. for 30 min and then kept in −20° C. Intracellular cAMP levels were detected using an alpha screen cAMP kit (6760635D from Perkin Elmer). The numerical values in the table are given as % increase over the basal level.

### Biological Example 3

³H-CGP 12177 Whole Cell β₂ Binding

Chinese Hamster Ovary (CHO) cells stably expressing the human β₂-adrenoceptor were grown in Dulbecco's modified Eagle's medium nutrient mix F12 (DMEM/F12) containing 10% fetal calf serum and 2 mM L-glutamine in a 37° C. humidified 5% CO₂:95% air atmosphere. The cells were seeded (100 000 cells/mL) into white-sided, clear-bottomed 96-well isoplates and allowed to grow to confluence for the following day's experiment. Next day, the media was removed from each well of the isoplate. Aliquots of the test compounds (10⁻² M in DMSO) were diluted in DMEM/F12 containing 2 mM L-glutamine (serum-free media) to final concentration ranges of 10⁻⁴ M to 10⁻¹⁰ M and added to each well. The radioligand ³H-CGP 12177 was diluted in the same medium (˜1 nM final well concentration) and added to each well. Propranolol and CGP 12177 (10 μM) were used to measure non-specific binding. The cells were incubated for 2 hours at 37° C. After incubation, the cells were washed twice by the addition and removal of 200 μL ice-cold phosphate-buffered saline (PBS). Cells were then lysed with Optiphase Supermix cocktail (100 μL/well) and a clear sealant top was applied to the plate. The plate was left overnight in the dark at room temperature, centrifuged (1000 rpm, 1 min) and measured in a MicroBeta2 Microplate Counter (Perkin Elmer) (10 minutes pre-incubation, 2 min counts/well).

All data points on each binding curve were measured in duplicates. Each 96-well plate also contained four determinations of total and non-specific binding. Non-specific binding was determined in the presence of CGP 12177 and propranolol (10 μM). A pre-determined K_{D}-value for the competing radioligand was used, obtained from a saturation binding measurement were increasing concentrations of the test compounds were used until the specific binding of ³H-CGP 12177 was completely inhibited. All data points are measured in counts per minute (CPM) and converted into disintegrations per minute (DPM) by dividing the count rate (CPM) with the counting efficiency of the instruments detector (57%). The numerical values were obtained by using the GraphPad Prism 7 programme and are given as -log K (pKᵢ) in the table.

Using the assays described in Biological Examples 1, 2 and 3, the following values were obtained.

| | Biological | Biological | Biological |
|---|---|---|---|
| Ex | example 1 | example 2 | example 3 |
| 1 | 50 | 1 | 4.6 |
| 2 | 27 | 3 | 5.2 |
| 3 | 86 | 18 | 4.6 |
| 4 | 62 | 40 | 6.0 |
| 5 | 39 | 18 | 5.3 |
| 6 | 23 | 3 | 5.0 |
| 7 | 23 | 1 | 5.3 |
| 8 | 12 | 15 | 4.7 |
| 9 | 18 | 4 | 4.3 |
| 10 | 39 | 5 | 4.6 |
| 11 | 50 | 10 | 4.4 |
| 12 | 40 | 190 | 5.1 |
| 13 | 42 | −1 | 4.8 |
| 14 | 17 | 20 | 4.8 |
| 15 | 31 | 20 | 6.1 |
| 16 | 23 | −56 | 5.3 |
| 17 | 26 | 70 | 5.2 |
| 18 | 26 | −23 | 5.2 |
| 19 | 30 | −52 | 6.3 |
| 20 | 12 | −19 | 5.5 |
| 21 | 37 | −16 | 5.7 |
| 22 | 30 | 20 | 4.9 |
| 23 | 28 | nt | 5.1 |
| 24 | 22 | nt | 5.0 |
| 25 | 32 | 115 | 4.4 |
| 26 | 10 | −0.3 | 4.6 |
| 27 | 6 | 86 | 5.9 |
| 28 | 39 | 2 | 6.4 |
| 29 | 48 | 222 | 6.5 |
| 30 | 50 | 231 | 7.1 |
| 31 | 36 | 439 | 7.5 |
| 32 | 53 | 371 | 5.2 |
| 33 | 55 | 1191 | 6.0 |
| 34 | 20 | 0 | 5.5 |
| 35 | 65 | 6536 | 5.4 |

nt = not tested

1. A compound of formula I 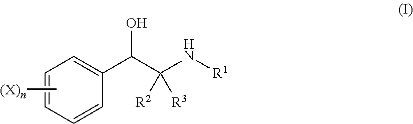
or a pharmaceutically acceptable salt thereof, for use in the treatment of hyperglycaemia or a disorder characterized by hyperglycaemia, wherein:
R¹ represents C₄₋₁₂ alkyl optionally substituted by one or more halo;
R² and R³ each independently represent H or C₁₋₃ alkyl optionally substituted by one or more halo;
or R² and R³ may be linked together to form, together with the carbon atom to which they are attached, a 3- to 6-membered ring, which ring optionally is substituted by one or more groups independently selected from halo and C₁ alkyl optionally substituted by one or more halo;
each X independently represents halo, R^{a}, —CN, —N₃, —N(R^{b})R^{c}, —NO₂, —ONO₂, —OR^{d}, —S(O)ₚR^{e} or —S(O)_{q}N(R^{f})R^{g};
R^{a} represents C₁₋₆ alkyl optionally substituted by one or more groups independently selected from G;
each R^{b}, R^{c}, R^{d}, R^{e}, R^{f} and R_{g} independently represents H or C₁₋₆ alkyl optionally substituted by one or more groups independently selected from G;
or alternatively any of R^{b} and R^{c} and/or R^{f} and R^{g} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
G represents halo, —CN, —N(R^{a1})R^{b1}, —OR^{c1}, —S(O)ₚR^{d1}, —S(O)_{q}N(R^{e1})R^{f1} or ═O;
each R^{a1}, R^{b1}, R^{c1}, R^{d1}, R^{e1} and R^{f1} independently represents H or C₁₋₆ alkyl optionally substituted by one or more halo;
or alternatively any of R^{a1} and R^{b1} and/or R^{e1} and R^{f1} may be linked together to form, together with the nitrogen atom to which they are attached, a 4- to 6-membered ring, which ring optionally contains one further heteroatom and which ring optionally is substituted by one or more groups independently selected from halo, C¹⁻³ alkyl optionally substituted by one or more halo, and ═O;
n represents 0 to 5;
each p independently represents 0, 1 or 2; and
each q independently represents 1 or 2.

2. The compound for use according to claim 1, wherein R¹ represents C₄₋₁₀ alkyl optionally substituted by one or more F.

3. The compound for use according to any preceding claim, wherein R¹ represents n-butyl, sec-butyl, tent-butyl, 2-pentyl, cyclopentyl, —CH₂-cyclopropyl, —(CH₂)₂-cyclopropyl, n-hexyl, —(CH₂)₃-cyclopropyl, —CH₂-cyclohexyl, n-octyl, —(CH₂)₂-cyclohexyl, —(CH₂)₃-cyclohexyl, 4,4,4-trifluorobutyl or 1-adamantyl.

4. The compound for use according to any preceding claim, wherein R² represents H and R³ represents H or methyl.

5. The compound for use according to any preceding claim, wherein R² and R³ each represent H.

6. The compound for use according to any preceding claim, wherein:
each X independently represents halo, R^{a}, —CN, —N₃, —N(R^{b})R^{c}, —NO₂ or OR^{d}, wherein R^{a} represents C₁₋₄ alkyl optionally substituted by one or more F, and R^{b}, R^{c} and R^{d} each independently represent H or C₁₋₄ alkyl optionally substituted by one or more F.

7. The compound for use according to any preceding claim, wherein each X independently represents F, Cl, R^{a}, —NH₂ or —OH, wherein R^{a} represents C₁₋₂ alkyl optionally substituted by 1 or more F.

8. The compound for use according to any preceding claim, wherein each X independently represents Cl, —NH₂, —CF₃ or —OH.

9. The compound for use according to any preceding claim, wherein n represents 0, 1, 2 or 3.

10. The compound for use according to any preceding claim, wherein n represents 3, and each X independently represents F, Cl, —NH₂, —CF₃ or —OH.

11. The compound for use according to any preceding claim, wherein n represents 3, and each X independently represents Cl, —NH₂, —CF₃ or —OH.

12. The compound for use according to claim 11, wherein the X groups are located in the 3-, 4- and 5-positions.

13. The compound for use according to any one of claims 1 to 9, wherein n represents 2, and each X independently represents F, Cl, —NH₂ or —OH.

14. The compound for use according to any one of claim 1 to 10 or 13, wherein n represents 2, and each X independently represents Cl or —OH.

15. The compound for use according to claim 13 or claim 14, wherein the X groups are located in the 3- and 4-positions, or the 3- and 5-positions.

16. The compound for use according to any preceding claim, wherein n represents 1.

17. The compound for use according to claim 16, wherein X represents F, Cl, R^{a} or —OH, wherein R^{a} represents C₁₋₂ alkyl optionally substituted by one or more F.

18. The compound for use according to claim 16 or claim 17, wherein X represents Cl or —OH.

19. The compound for use according to any one of claims 16 to 18, wherein X is located in the 3- or the 4-position.

20. The compound for use according to any one of the preceding claims, wherein when n represents 1 and X is located in the 4-position and represents —OR^{d}, then R^{d} represents H.

21. The compound for use according to any one of claims 1 to 20, wherein the compound for use is a compound of formula IA 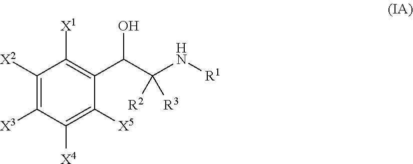
wherein:
R¹, R² and R³ are as defined in any preceding claim; and
X¹, X², X³, X⁴ and X⁵ each independently represent H or X, wherein X is as defined in any preceding claim.

22. The compound for use according to claim 21, wherein:
X¹ and X⁵ each represent H, fluoro or chloro; and
X², X³ and X⁴ each independently represent H, halo, R^{a}, —CN, —N₃, —N(R^{b})R^{c}, —NO₂ or —OR^{d};
wherein R^{a} represents C₁₋₄ alkyl optionally substituted by one or more F, R^{b}, R^{c} and R^{d} each independently represent H or C₁₋₄ alkyl optionally substituted by one or more F.

23. The compound for use according to claim 21 or claim 22, wherein:
X¹, X² and X⁵ each represent H; and
X³ and X⁴ each independently represent H, halo, R^{a}, —CN, —NH₂, or —OH, wherein R^{a} represents C¹⁻² alkyl optionally substituted by one or more F.

24. The compound for use according to any one of claims 21 to 23, wherein:
X¹, X² and X⁵ each represent H; and
X³ and X⁴ each independently represent H, halo, —NH₂, —CN or —OH.

25. The compound for use according to any one of claims 21 to 24, wherein:
X¹, X² and X⁵ each represent H; and
X³ and X⁴ each independently represent H, halo, —CN or —OH.

26. The compound for use according to any one of claims 21 to 25, wherein:
X¹, X² and X⁵ each represent H; and
X³ and X⁴ each independently represent H, F, Cl, —CN, —NH₂ or —OH.

27. The compound for use according to any one of claims 21 to 26, wherein:
X¹, X² and X⁵ each represent H; and
X³ and X⁴ each independently represent H, Cl or —OH.

28. The compound for use according to claim 21 or claim 22, wherein:
X¹ and X⁵ each represent H. X² and X⁴ each independently represent halo, R^{a}, —CN, —N₃, —N(R^{b})R^{c}, —NO₂ or —OR^{d};
wherein R^{a} represents C₁₋₄ alkyl optionally substituted by one or more F, and R^{b}, R^{c} and R^{d} each independently represents H or C₁₋₄ alkyl optionally substituted by one or more F; and
X³ represents H halo, R^{a}, —CN, —N₃, —N(R^{b})R^{c}, —NO₂ or —OR^{d}; wherein R^{a} represents C₁₋₄ alkyl optionally substituted by one or more F, and R^{b}, R^{c} and R^{d} each independently represents H or C₁₋₄ alkyl optionally substituted by one or more F

29. The compound for use according to claim 28, wherein:
X¹ and X⁵ each represent H;
X² and X⁴ each independently represent F, Cl, R^{a} or OR^{d}; wherein R^{a} represents C₁₋₂ alkyl optionally substituted by one or more F, and R^{d} represents H or C₁₋₂ alkyl optionally substituted by one or more F; and
X³ represents H, —N(R^{b})R^{c}or —OR^{d}; wherein R^{b}, R^{c} and R^{d} each independently represent H or C₁₋₂ alkyl optionally substituted by one or more F.

30. The compound for use according to claim 28 or claim 29, wherein:
X¹ and X⁵ each represent H;
X² and X⁴ each independently represent F, Cl, —CF₃ or -OH; and
X³ represents H, —NH₂ or —OH.

31. The compound for use as according to any one of claims 28 to 30, wherein:
X¹ and X⁵ each represent H;
X² and X⁴ each independently represent Cl, —CF₃ or —OH; and
X³ represents H, —NH₂ or —OH.

32. The compound for use according to any one of claims 1 to 31, wherein the compound of formula I or formula IA is a compound of formula IC 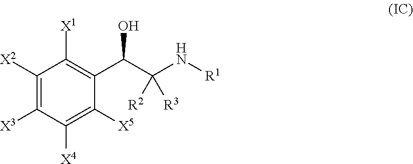
wherein X¹, X², X³, X⁴, X⁵, R¹, R² and R³ are as defined in any preceding claim.

33. The compound for use according to claim 32, wherein:
X¹, X³, X⁴ and X⁵ each represent H;
X³ represents —OH;
R¹ represents C₄ alkyl; and/or
R² and R³ both represent H.

34. The compound for use according to any preceding claim, wherein the compound is selected from the group consisting of:
4-(2-(butylamino)-1-hydroxyethyl)phenol,
(R)-4-(2-(butylamino)-1-hydroxyethyl)phenol,
2-(butylamino)-1-(3-chlorophenyl)ethan-1-ol,
4-(2-((cyclopropylmethyl)amino)-1-hydroxyethyl)phenol,
4-(2-(tert-butylamino)-1-hydroxyethyl)phenol,
4-(2-(sec-butylamino)-1-hydroxyethyl)phenol,
4-(2-((cyclohexylmethyl)amino)-1-hydroxyethyl)phenol,
4-(2-(cyclopentylamino)-1-hydroxyethyl)phenol,
4-(1-hydroxy-2-(pentan-2-ylamino)ethyl)phenol,
4-(2-(adamantan-1-ylamino)-1-hydroxyethyl)phenol,
2-((cyclohexylmethyl)amino)-1-phenylethan-1-ol,
4-(2-(butylamino)-1-hydroxypropyl)phenol,
4-(1-hydroxy-2-(pentan-2-ylamino)propyl)phenol,
3-(2-(butylamino)-1-hydroxyethyl)phenol,
3-(1-hydroxy-2-(pentan-2-ylamino)ethyl)phenol,
4-(2-((2-cyclohexylethyl)amino)-1-hydroxyethyl)phenol,
4-(2-(hexylamino)-1-hydroxyethyl)phenol,
4-(1-hydroxy-2-(octylamino)ethyl)phenol,
2-chloro-4-(1-hydroxy-2-(pentan-2-ylamino)ethyl)phenol,
4-(2-(butylamino)-1-hydroxyethyl)-2-chlorophenol,
4-(2-((3-cyclohexylpropyl)amino)-1-hydroxyethyl)phenol,
4-(2-((2-cyclopropylethyl)amino)-1-hydroxyethyl)phenol,
4-(2-((3-cyclopropylpropyl)amino)-1-hydroxyethyl)phenol,
4-(1-hydroxy-2-((4,4,4-trifluorobutyl)amino)ethyl)phenol,
5-(2-(butylamino)-1-hydroxyethyl)benzene-1,3-diol,
4-(2-(butylamino)-1-hydroxyethyl)-2,6-dichlorophenol,
2,6-dichloro-4-(1-hydroxy-2-(pentan-2-ylamino)ethyl)phenol,
1-(4-amino-3,5-dichlorophenyl)-2-(pentan-2-ylamino)ethan-1-ol,
1-(4-amino-3,5-dichlorophenyl)-2-(butylamino)ethan-1-ol,
1-(4-amino-3-chloro-5-(trifluoromethyl)phenyI)-2-(butylamino)ethan-1-ol,
1-(4-amino-3-chloro-5-(trifluoromethyl)phenyI)-2-(pentan-2-ylamino)ethan-1-ol,
5-(2-(butylamino)-1-hydroxyethyl)-2-chlorophenol,
2-chloro-5-(1-hydroxy-2-(pentan-2-ylamino)ethyl)phenol,
1-(3-amino-4-chlorophenyl)-2-(butylamino)ethan-1-ol, and
1-(4-amino-3,5-difluorophenyI)-2-(butylamino)ethan-1-ol, and pharmaceutically acceptable salts thereof.

35. The use of a compound as defined in any one of claims 1 to 34 for the manufacture of a medicament for the treatment of hyperglycaemia or a disorder characterized by hyperglycaemia.

36. A method of treating hyperglycaemia or a disorder characterized by hyperglycaemia comprising administering to a patient in need thereof a therapeutically effective amount of a compound as defined in any one of claims 1 to 34, or a pharmaceutically acceptable salt thereof.

37. A pharmaceutical composition for use in the treatment of hyperglycaemia or a disorder characterized by hyperglycaemia comprising a compound as defined in any one of claims 1 to 34, and optionally one or more pharmaceutically acceptable adjuvant, diluent and/or carrier.

38. The compound or composition for use, method or use according to any one of claims 1 to 37, wherein the treatment is of type 2 diabetes.

39. The compound or composition for use, method or use according to any one of claims 1 to 37, wherein the hyperglycaemia or disorder characterised by hyperglycaemia is, or is characterised by, the patient displaying severe insulin resistance.

40. The compound or composition for use, method or use according to any one of claim 1 to 37 or 39, wherein the disorder characterised by hyperglycaemia is selected from the group consisting of Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

41. A combination product comprising:
(a) a compound as defined in any one of claims 1 to 34; and
(b) one or more other therapeutic agent that is useful in the treatment of hyperglycaemia or a disorder characterized by hyperglycaemia,
wherein each of components (a) and (b) is formulated in admixture, optionally with one or more a pharmaceutically-acceptable adjuvant, diluent or carrier.

42. A kit-of-parts comprising:
(a) a pharmaceutical composition comprising a compound as defined in any one of claims 1 to 34, and optionally one or more pharmaceutically acceptable adjuvant, diluent and/or carrier, and
(b) one or more other therapeutic agent that is useful in the treatment of hyperglycaemia or a disorder characterized by hyperglycaemia, optionally in admixture with one or more pharmaceutically-acceptable adjuvant, diluent or carrier,
which components (a) and (b) are each provided in a form that is suitable for administration in conjunction with the other.

43. A compound selected from the group consisting of:
4-(2-((cyclopropylmethyl)amino)-1-hydroxyethyl)phenol,
4-(1-hydroxy-2-(pentan-2-ylamino)propyl)phenol,
3-(1-hydroxy-2-(pentan-2-ylamino)ethyl)phenol,
4-(2-((2-cyclohexylethyl)amino)-1-hydroxyethyl)phenol,
4-(1-hydroxy-2-(octylamino)ethyl)phenol,
2-chloro-4-(1-hydroxy-2-(pentan-2-ylamino)ethyl)phenol,
4-(2-(butylamino)-1-hydroxyethyl)-2-chlorophenol,
4-(2-((3-cyclohexylpropyl)amino)-1-hydroxyethyl)phenol,
4-(2-((2-cyclopropylethyl)amino)-1-hydroxyethyl)phenol,
4-(2-((3-cyclopropylpropyl)amino)-1-hydroxyethyl)phenol,
4-(1-hydroxy-2-((4,4,4-trifluorobutyl)amino)ethyl)phenol,
4-(2-(butylamino)-1-hydroxyethyl)-2,6-dichlorophenol,
2,6-dichloro-4-(1-hydroxy-2-(pentan-2-ylamino)ethyl)phenol,
1-(4-amino-3,5-dichlorophenyl)-2-(pentan-2-ylamino)ethan-1-ol,
1-(4-amino-3-chloro-5-(trifluoromethyl)phenyl)-2-(butylamino)ethan-1-ol, and
1-(4-amino-3-chloro-5-(trifluoromethyl)phenyl)-2-(pentan-2-ylamino)ethan-1-ol,
5-(2-(butylamino)-1-hydroxyethyl)-2-chlorophenol,
2-chloro-5-(1-hydroxy-2-(pentan-2-ylamino)ethyl)phenol, and
1-(4-amino-3,5-difluorophenyI)-2-(butylamino)ethan-1-ol, and a pharmaceutically acceptable salts thereof.

44. A compound selected from the group consisiting of:
2-(butylamino)-1-(3-chlorophenyl)ethan-1-ol,
4-(2-((cyclopropylmethyl)amino)-1-hydroxyethyl)phenol,
4-(2-((cyclohexylmethyl)amino)-1-hydroxyethyl)phenol,
4-(2-(adamantan-1-ylamino)-1-hydroxyethyl)phenol,
2-((cyclohexylmethyl)amino)-1-phenylethan-1-ol,
4-(2-(butylamino)-1-hydroxypropyl)phenol,
3-(1-hydroxy-2-(pentan-2-ylamino)ethyl)phenol,
3-(1-hydroxy-2-(pentan-2-ylamino)ethyl)phenol,
4-(2-((2-cyclohexylethyl)amino)-1-hydroxyethyl)phenol,
4-(1-hydroxy-2-(octylamino)ethyl)phenol,
2-chloro-4-(1-hydroxy-2-(pentan-2-ylamino)ethyl)phenol,
4-(2-(butylamino)-1-hydroxyethyl)-2-chlorophenol,
4-(2-((3-cyclohexylpropyl)amino)-1-hydroxyethyl)phenol,
4-(2-((2-cyclopropylethyl)amino)-1-hydroxyethyl)phenol,
4-(2-((3-cyclopropylpropyl)amino)-1-hydroxyethyl)phenol,
4-(1-hydroxy-2-((4,4,4-trifluorobutyl)amino)ethyl)phenol,
4-(2-(butylamino)-1-hydroxyethyl)-2,6-dichlorophenol,
2,6-dichloro-4-(1-hydroxy-2-(pentan-2-ylamino)ethyl)phenol,
1-(4-amino-3,5-dichlorophenyl)-2-(pentan-2-ylamino)ethan-1-ol,
1-(4-amino-3-chloro-5-(trifluoromethyl)phenyl)-2-(butylamino)ethan-1-ol,
1-(4-amino-3-chloro-5-(trifluoromethyl)phenyl)-2-(pentan-2-ylamino)ethan-1-ol,
5-(2-(butylamino)-1-hydroxyethyl)-2-chlorophenol,
2-chloro-5-(1-hydroxy-2-(pentan-2-ylamino)ethyl)phenol,
1-(3-amino-4-chlorophenyl)-2-(butylamino)ethan-1-ol, and
1-(4-amino-3,5-difluorophenyl)-2-(butylamino)ethan-1-ol, and pharmaceutically acceptable salts thereof, for use in medicine.

45. A pharmaceutical composition comprising a compound as defined in claim 43 or claim 44, and optionally one or more pharmaceutically acceptable adjuvant, diluent and/or carrier.

46. A compound, composition, kit, combination product, composition for use, compound for use, use or method substantially as described herein, with reference to the examples.
